(19) United States
(12) Patent Application Publication
(10) Pub. No.: US 2013/0190487 A1
(43) Pub. Date: Jul. 25, 2013

(54) PROCESS FOR THE PREPARATION OF EZETIMIBE AND DERIVATIVES THEREOF

(71) Applicants: Anton Stimac, Ljubljana (SL); Barbara Mohar, Grosuplje (SL); Michel Stephan, Vanves (FR); Mojca Bevc, Mirna (SL); Rok Zupet, Ljubljana (SL); Andrej Gartner, Ljubljana (SL); Vesna Kroselj, Sentjernej (SL); Matej Smrkolj, Trbovlje (SL); Davor Kidemet, Varazdin (HR); Gregor Sedmak, Ljubljana (SL); PRIMOZ BENKIC, LJUBLJANA (SL); ALEN KLJAJIC, CELJE (SL); MIHA PLEVNIK, IVANCNA GORICA (SL)

(72) Inventors: Anton Stimac, Ljubljana (SL); Barbara Mohar, Grosuplje (SL); Michel Stephan, Vanves (FR); Mojca Bevc, Mirna (SL); Rok Zupet, Ljubljana (SL); Andrej Gartner, Ljubljana (SL); Vesna Kroselj, Sentjernej (SL); Matej Smrkolj, Trbovlje (SL); Davor Kidemet, Varazdin (HR); Gregor Sedmak, Ljubljana (SL); PRIMOZ BENKIC, LJUBLJANA (SL); ALEN KLJAJIC, CELJE (SL); MIHA PLEVNIK, IVANCNA GORICA (SL)

(73) Assignee: KRKA, NOVO MESTO (SL)

(21) Appl. No.: 12/524,346

(22) PCT Filed: Jan. 24, 2008
(86) PCT No.: PCT/EP08/00546

§ 371 Date: Dec. 7, 2009

(30) Foreign Application Priority Data
Jan. 24, 2007 (EP) 07001537.5
Aug. 1, 2007 (EP) 07015107.1
Oct. 12, 2007 (EP) 07020070.4
Dec. 6, 2007 (EP) 07023686.4
Dec. 17, 2007 (EP) 07024430.6

(51) Int. Cl. C07D 205/08

(52) U.S. Cl. CPC C07D 205/08
USPC 540/200

(57) ABSTRACT

The present invention relates to the method of preparing of ezetimibe and in particular to novel intermediates for its synthesis and an improved process for preparing such intermediates. Said intermediates may be obtained in high yields and purity in a fast and cost efficient manner. The present invention relates to a novel crystalline form of ezetimibe as well.

The present invention relates to an improved process for the preparation of 1-(4-fluorophenyl)-3(R)-[3-(4-fluorophenyl)-3(S)-hydroxypropyl]-4(S)-(4-hydroxyphenyl)-2-azetidinone, based on [ruthenium-R³R⁴NSO₂-1,2-diamine] catalyzed asymmetric transfer hydrogenation of p-fluoroacetophenones. Said intermediates may be obtained in high yields and purity in a fast and cost efficient manner.

Hypercholesterolemia and high blood- or plasma-cholesterol are common diseases in the well-situated countries. Hypercholesterolemia has been implicated in atherosclerosis, hardening-of-arteries, heart-attack, and is one of several conditions that may lead to coronary and artery diseases. The risk group includes the overweight, smokers, those with a poor diet (e.g. one rich in saturated fats), those who take inadequate exercise and suffering from stress. For such risk individuals, as well as those tested and found to have unduly high plasma cholesterol levels, a variety of treatments have been proposed, e.g. changes in diet and habits, increased exercise, etc. However such treatments are not always easy to enforce and therefore there exists a constant need for medicinal treatments which have been effective at reducing plasma cholesterol levels.

Statins (e.g. fluvastatin, simvastatin, lovastatin, atorvastatin, rosuvastatin), and in particular simvastatin, are commonly used in the treatment or prevention of high cholesterol level in individuals. Also other compounds having a different mode of action with regard to a reduction of blood cholesterol levels have been proposed for use. Among them is a well known drug ezetimibe, a class of lipid-lowering compounds that selectively inhibits the intestinal absorption of cholesterol and related phytosterols.

Ezetimibe with chemical name 1-(4-fluorophenyl)-3(R)-[3-(4-fluorophenyl)-3(S)-hydroxypropyl]-4(S)-(4-hydroxyphenyl)-2-azetidinone and identified by the structure formula (Ia) was disclosed in EP 0720599.

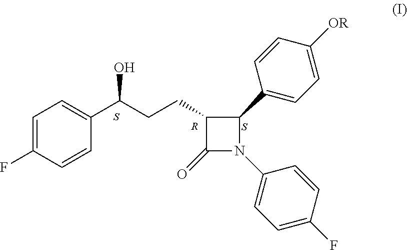

R═H Ezetimibe structural formula Ia:

The mechanism of absorption and resorption inhibition of cholesterol of ezetimibe involves increased excretions of cholesterol and its intestinally generated metabolites with the faeces. This effect results in lowered body cholesterol levels, increased cholesterol synthesis, and decreased triglyceride synthesis. The increased cholesterol synthesis initially provides for the maintenance of cholesterol levels in the circulation, levels that eventually decline as the inhibition of cholesterol absorption and resorption continues. The overall effect of the drug action is the lowering of cholesterol level in the circulation and tissues of the body. Ezetimibe is also suspected to reduce plasma concentrations of the noncholesterol sterols sitosterol and campesterol, suggesting an effect on the absorption of these compounds as well.

Different synthetic routes to ezetimibe and derivatives thereof have been described in literature wherein the key-step relies on the asymmetric reduction of α-functionalized p-fluoroacetophenone intermediates with the general formula (IIa), (IIb), (IIIb) or (IV).

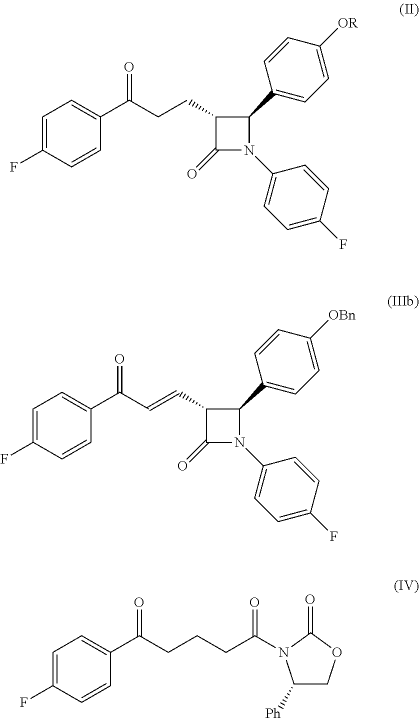

wherein:

R═H (IIa):

R=Bn (IIb):

In EP 0 906 278 and EP 0 720 599, ezetimibe (Ia) and its derivative (Ib) (formula (I) wherein R=Bn) were prepared through borane reduction of the corresponding ketone (IIa) and (IIb). The reduction was catalyzed by 10 mol % of (R)-tetrahydro-1-methyl-3,3-diphenyl-1H,3H-pyrrolo-[1,2-c][1,3,2]oxazaborolidine at −20° C. followed by O-debenzylation for (Ib). The same type of reduction was applied to ketones (IIIb) and (IV) (disclosed in EP 0 707 567). According to this literature, the alcohols obtained from reduction of (IIa), (IIb) or (IV) were isolated in 70 to 80% yield with a diastereomeric ratio (dr) of 96:4 to 99:1. The reduction of compound with the general formula (IIIb) led to a dr of 88:12. These processes generate a stoichoimetric amount of borate waste salts.

An alternative synthesis of ezetimibe, a microbial reduction with high dilution of ketone (IIa) to ezetimibe as a single diastereomer was described in EP 1 169 468. However, this process is low yielding (15% yield).

Furthermore, the intermediate ketone (IIb) used in the above mentioned literature is syrupy and it can only be obtained pure after a tedious chromatographic purification.

On the another hand, alternative means of stereoselective reduction of arylketones excluding the stoichiometric use of borane reagents have been disclosed. \check{S}terk et al. (Tetrahedron: Asymmetry 2002, 13, 2605-2608) performed the efficient asymmetric transfer hydrogenation of various classes of ketones using formic acid/triethylamine mixture catalyzed by optically pure ruthenium or rhodium complexes of N—(N,N-dialkylsulfamoyl)-1,2-diamine ligands. The catalysts can be prepared in situ and do not require any special inert gas manipulation.

Polymorphic forms of ezetimibe are described for example in WO 2005/009955, WO 2005/062897, WO 2006/060808, US 2006/0234996, IPCOM000131677D disclosing mainly anhydrous and hydrous crystalline forms, different mixtures thereof and amorphous form of ezetimibe. The obtained polymorphic form depends on the solvent used in the recrystallization step and on water content of the final product (WO 2006/060808). Ezetimibe form A, form B and the process for the preparation thereof is disclosed in WO 2006/060808. The solvated forms of ezetimibe form B are disclosed in IPCOM000131677D.

Accordingly, there is a need in the art to provide an alternative synthesis of ezetimibe permitting the provision of higher yields of said compound with higher purity and obtained in a cost intensive overall synthesis.

The above mentioned problem has been solved by providing novel intermediates of ezetimibe and a modified reaction scheme allowing the provision of said intermediates as well as the end product in higher optical purity and higher yield.

### SUMMARY OF THE INVENTION

The present invention relates to a process for preparing a compound represented by general formula

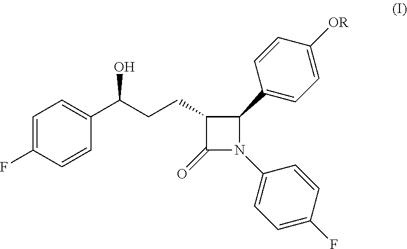

wherein

R represents a hydrogen atom, a protective group selected from the group consisting of trisubstituted silyl, arylmethyl, tetrahydro-2H-pyranyl, mono or disubstituted arylmethyl with the substituents, selected from the group consisting of halides, methoxy, nitro, phenyl, naphthyl and any combinations thereof,

comprising the steps of

a) metal-catalysed asymmetric transfer hydrogenation of p-fluoroacetophenones of general formula (II)

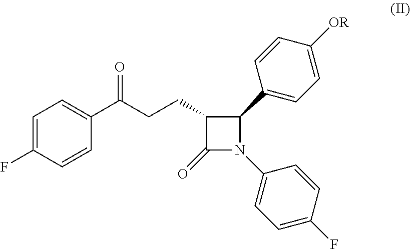

wherein R has the same meaning as above

by using of a hydrogen donor in the presence of a metal catalyst based on Ruthenium complexes,

b) obtaining the compound represented by general formula (I), and

c) optionally purifying the compound represented by general formula (I).

The synthetic route for the preparation of ezetimibe and derivatives thereof and novel intermediates according to the present invention are presented in Schemes 1-6.

In another embodinment the present invention relates to the use of Ruthenium catalyst [(S,S)—N-(piperidyl-N-sulfonyl)-1,2-diphenylethylenediamine](η⁶-mesitylene)ruthenium in the preparation of compound of formula (I) as defined above.

In another embodiment, the present invention provides novel crystalline form S ezetimibe and the process for its preparation as defined in the accompanying claims. Ezetimibe form S is specified by an X-ray powder diffraction pattern, by ¹H-NMR and by ¹³C-NMR. Ezetimibe form S is characterized by a water content of about 0 to about 2%, determined by Karl Fischer analysis, by purity more than 90% and by content of tert-butanol from about 8 to about 15%. Ezetimibe form S has a particle size of less than about 100 microns. Ezetimibe form S contains not more than 20%, of other polymorphic forms.

In another embodiments, the present invention provides a pharmaceutical composition containing ezetimibe prepared according to the process of the present invention and as defined in the accompanying claims.

### BRIEF DESCRIPTION OF THE FIGURES

FIG. 1: Powder X-ray diffraction pattern of hydrated form H

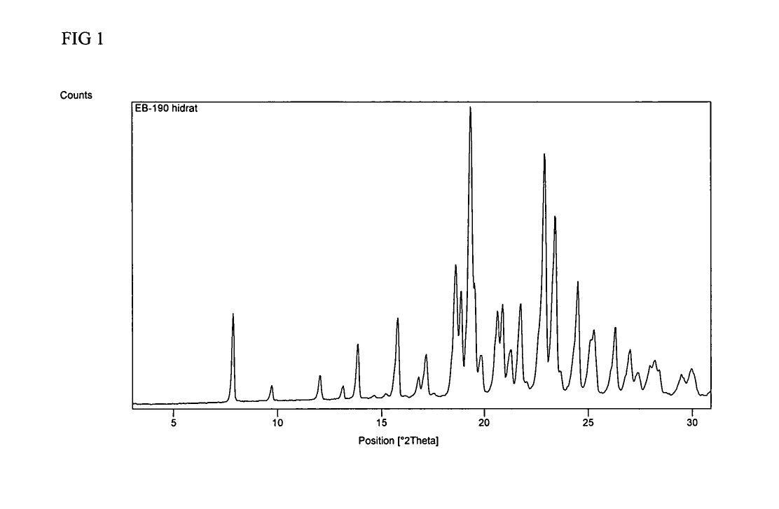

FIG. 2: Powder X-ray diffraction pattern of anhydrous form A

FIG. 3: Powder X-ray diffraction pattern of form S

FIG. 4: NMR spectra of form S

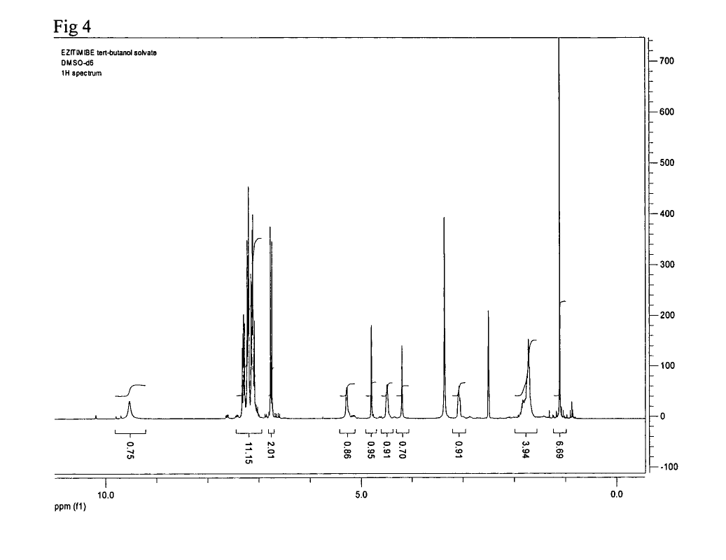

FIG. 5: Form S as seen through a microscope with different magnitudes

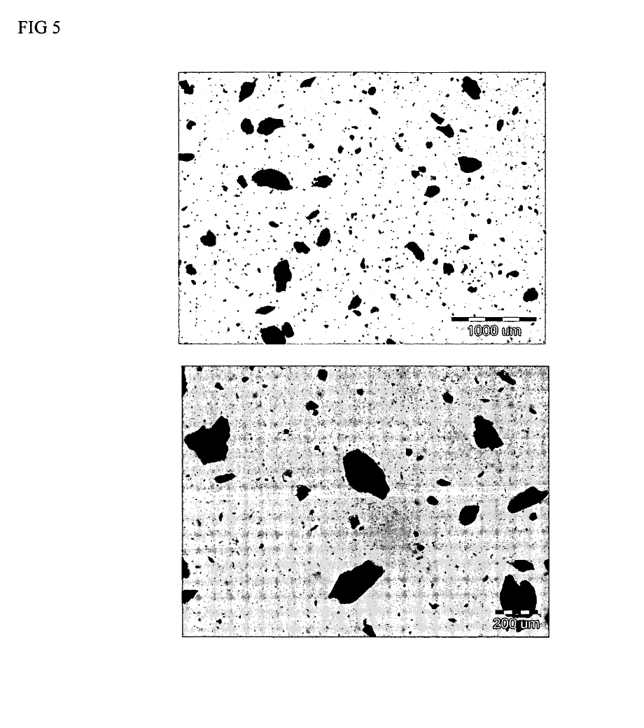

FIG. 6: Powder X-ray diffraction pattern of Methyl 3-[(2S,3R)-1-(4-fluorophenyl)-2-(4-hydroxyphenyl)-4-oxoazetidinyl]propionate

FIG. 7: Powder X-ray diffraction pattern of Methyl 3-{(3R,4S)-1-(4-fluorophenyl)-2-oxo-4-[4-(trityloxy)phenyl]azetidin-3-yl}propionate

FIG. 8: Powder X-ray diffraction pattern of Methyl 3-{(2S,3R)-2-[4-(benzyloxy)phenyl]-1-(4-fluorophenyl)-4-oxoazetidin-3-yl}propionate

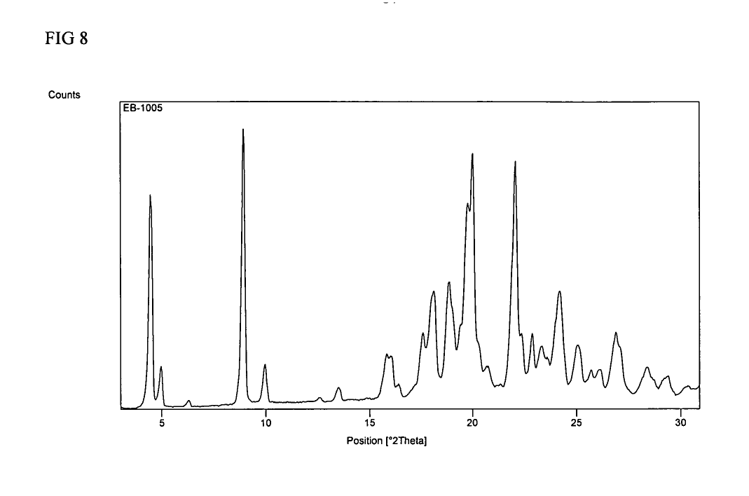

FIG. 9: Anhydro form A as seen through a microscope

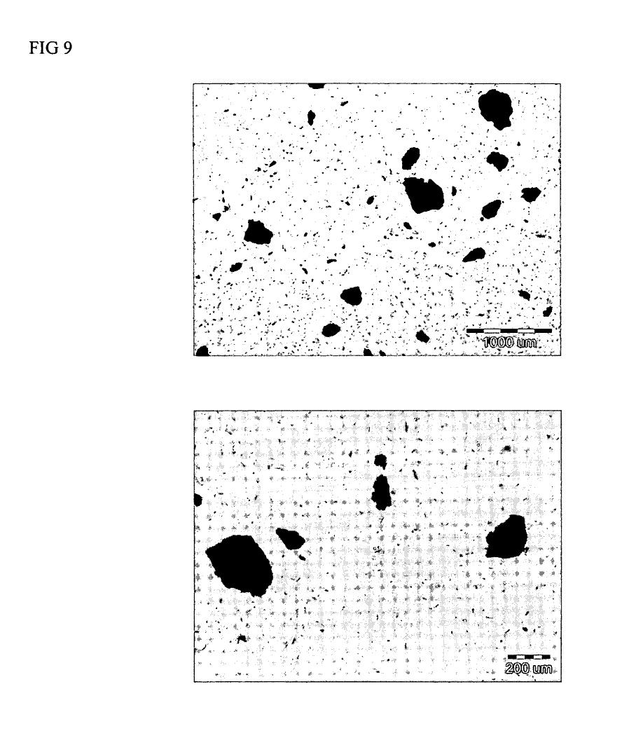

FIG. 10: Hydrated form H as seen through a microscope

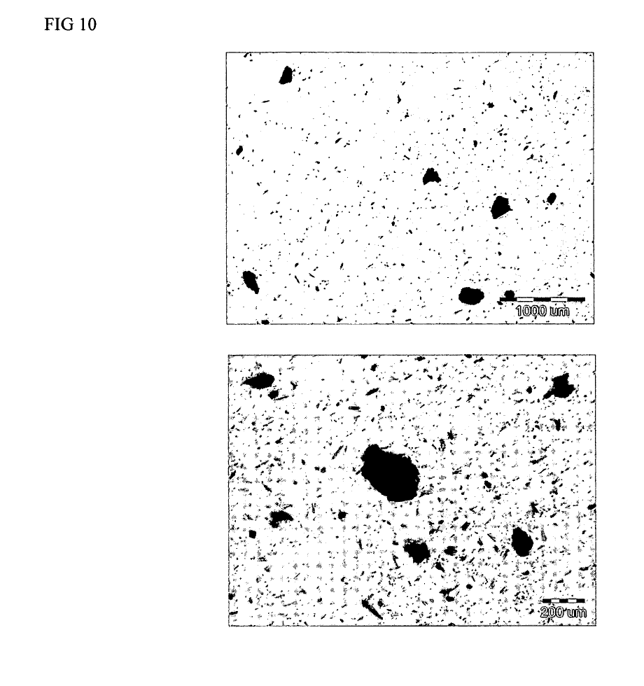

FIG. 11: Comparison of dissolution profiles of ezetimibe from Ezetimibe 10 mg tablets. The dissolution profiles were prepared by using as dissolution media: 0.1 M HCl with Tween, 900 ml; employing as dissolution apparatus: Apparatus 2—paddle (Ph.Eur. and USP).

FIG. 12: Morphology of ezetimibe particles obtained by de-solvation of ezetimibe tert-butanol solvate, i.e. form S, in mixture of water and isopropanol

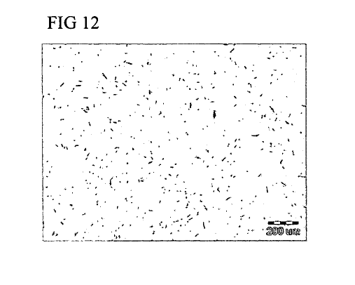

FIG. 13: Morphology of ezetimibe particles obtained by de-solvation of ezetimibe tert-butanol solvate, i.e. form S, in water

FIG. 14: Morphology of ezetimibe particles obtained by de-solvation of ezetimibe tert-butanol solvate, i.e. form S, in an anhydrous solvent.

### DETAILED DESCRIPTION OF THE INVENTION

According to a preferred embodiment of the present invention a process for preparing a compound represented by general formula

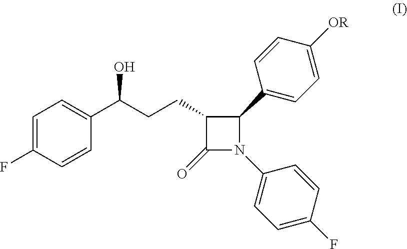

is provided, wherein R represents a hydrogen atom, a protective group selected from the group consisting of trisubstituted silyl, arylmethyl, tetrahydro-2H-pyranyl, mono or disubstituted arylmethyl with the substituents, selected from the group consisting of halides, methoxy, nitro, phenyl, naphthyl and any combinations thereof. Said process (Scheme 6) comprises the steps of

a) metal-catalysed asymmetric transfer hydrogenation of p-fluoroacetophenones of general formula (II)

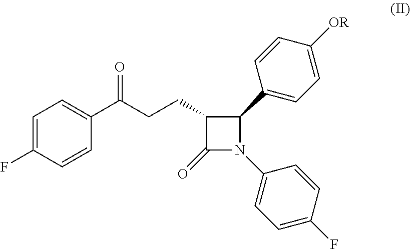

wherein R has the same meaning as above, i.e. R represents a hydrogen atom, a protective group selected from the group consisting of trisubstituted silyl, arylmethyl, tetrahydro-2H-pyranyl, mono or disubstituted arylmethyl with the substituents, selected from the group consisting of halides, methoxy, nitro, phenyl, naphthyl and any combinations thereof. The transfer hydrogenation is performed by using of a hydrogen donor in the presence of a metal catalyst based on Ruthenium complexes;

b) obtaining the compound represented by general formula (I), preferably with a diastereomeric ratio (dr) of more than 99:1; and

c) optionally purifying the compound represented by general formula (I).

According to an embodiment of the present invention R is selected from the group consisting of t-butyldimethylsilyl, t-butyldiphenylsilyl, triisopropylsilyl, trityl, benzyl, p-bromobenzyl, p-chlorobenzyl, p-nitrobenzyl, o-nitrobenzyl, p-phenylbenzyl, p-methoxybenzyl, tetrahydro-2H-pyranyl, characterised in that said process provides the compound represented by general formula (I) with a diastereometric ratio of more than 99:1.

According to another embodiment, R is selected from the group consisting of p-bromobenzyl, p-chlorobenzyl, p-nitrobenzyl, p-methoxybenzyl, trityl, tert-butyldimethylsilyl, tetrahydro-2H-pyranyl and benzyl.

According to the present invention, the process relies on the use of a hydrogen donor in the presence of a metal catalyst based on ruthenium complexes of optically active N-sulfamoyl-1,2-diamine ligands (R³R⁴NSO₂-1,2-diamine) of the general formula (VI):

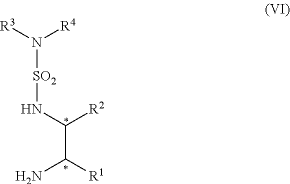

wherein:
C* represents an asymmetric carbon atom;
R¹ and R² independently represent a hydrogen atom, an optionally substituted aryl, or cycloalkyl, or R¹ and R² may be linked together to form a cyclohexane ring;
R³ and R⁴ independently represent a hydrogen atom, a C₁₋₁₅ alkyl, linear or branched, optionally substituted with an aryl; preferably, R³ and/or R⁴ can be selected from the group consisting of methyl, iso-propyl, cyclohexyl; or R³ and R⁴ may be linked together to form with the nitrogen atom an optionally substituted C₄₋₆ ring such as e.g. pyrrolidyl, piperidyl, morpholyl, or azepanyl.

According to an embodiment, the optically active N-sulfamoyl-1,2-diamine ligands have enantiomeric excess more than 99%.

According to an embodiment, R³ and/or R⁴ can be selected from the group consisting of methyl, iso-propyl and cyclohexyl.

According to another embodiment, R³ and R⁴ are linked together to form a ring selected from the group consisting of pyrrolidyl, piperidyl, morpholyl and azepanyl.

Preferably the Ruthenium complex is represented by the formula:

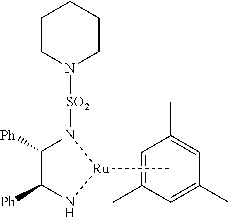

[(S,S)—N-(piperidyl-N-sulfonyl)-1,2-diphenylethylenediamine](η⁶-mesitylene)ruthenium (Abbreviated: [Ru(mesitylene)(S,S)-piperidyl-SO₂-DPEN])

Yet another embodiment of the present invention is the use of Ruthenium complex [(S,S)—N-(piperidyl-N-sulfonyl)-1,2-diphenylethylenediamine](η⁶-mesitylene)ruthenium in the preparation of compound of formula (I)

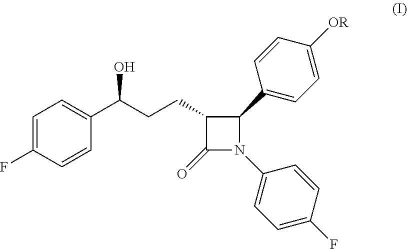

by asymmetric transfer hydrogenation of p-fluoroacetophenones of general formula (II)

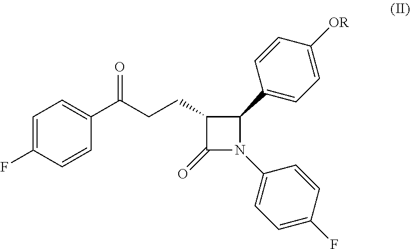

wherein R is selected from the group consisting of a hydrogen atom, a protective group selected from the group consisting of trisubstituted silyl, arylmethyl, tetrahydro-2H-pyranyl, mono or disubstituted arylmethyl with the substituents, selected from the group consisting of halides, methoxy, nitro, phenyl, naphthyl and any combinations thereof.

The optically active ruthenium complex is prepared from a ruthenium metal precursor and an optically active (preferably >99% ee) N-sulfamoyl-1,2-diamine ligand of the general formula (VI) (wherein R¹, R², R³ and R⁴ are as defined above) and is used either in an isolated form or in situ. The ruthenium metal precursor consists of η⁶-arene-ruthenium(II) halide dimers of formula [RuX₂(η⁶-arene)]₂, wherein η⁶-arenerepresents an arene, selected from the group consisting of benzene, p-cymene, mesitylene, 1,3,5-triethylbenzene, hexamethylbenzene, anisole, and wherein X is a halide selected from the group consisting of chloride, bromide and iodide.

The Ruthenium catalyst used in the metal-catalysed asymmetric transfer hydrogenation according to the present invention can be obtained from the Ruthenium complex by activation in the presence of a base and/or the hydrogen donor.

The metal-catalyzed asymmetric transfer hydrogenation according to the present invention can be carried out in the presence of a hydrogen donor known from literature as for example in Palmer et al. Tetrahedron: Asymmetry 1999, 10, 2045-2061. Preferably used are derivatives of HCO₂H such as e.g. HCO₂H-Et₃N, HCO₂H-iso-Pr₂NEt, HCO₂H-metal bicarbonates, HCO₂H-metal carbonates (the metal is selected from the group consisting of Na, K, Cs, Mg, Ca) and the like.

Suitable solvents for the process of the present invention include but are not limited to solvents such as dichloroethane, acetonitrile, N,N-dimethylformamide (DMF), N,N-dimethylacetamide (DMA), 1-methyl-2-pyrrolidinone (NMP), 1,1,3,3-tetramethylurea (TMU), 1,3-dimethyl-2-imidazolidinone (DMEU) N,N′-dimethylpropyleneurea (DMPU) and mixtures thereof.

The metal-catalysed asymmetric transfer hydrogenation may be conducted at reaction temperatures from about 15° C. to about 70° C., preferably between about 30° C. to about 40° C.

Surprisingly we found that the required amount of Ruthenium catalyst used in the process according to the present invention is low in comparison to the amount of other catalysts used in the syntheses of ezetimibe known from the prior art. The ruthenium catalyst may be used in an amount varying from about 0.05 to about 10 mol %, preferably between about 0.1 and about 1.0 mol %.

According to a preferred embodiment, the compound of formula (I) is ezetimibe.

The starting material used in the process of the present invention (Scheme 1), which may be compound of general formula (Vb; Z═CO₂Me), may be hydrogenated in the presence of 10% Pd—C to yield the hydroxy deprotected compound of general formula (Va; Z═CO₂Me), which is further protected by a variety of reagents. The products of formula (Va; Z═CO₂Me), (Vb; Z═CO₂Me) and the O-trityl derivative of formula (Vh; Z═CO₂Me) are crystalline and are characterized by powder X-ray diffraction peaks:

| Va; Z = CO₂Me, | Vb; Z = CO₂Me, | Vh; Z = CO₂Me, |
|---|---|---|
| R = H | R = CH₂Ph | R = CPh₃ |
| (°2Θ) | (°2Θ) | (°2Θ) |
| 9.3 | 4.5 | 5.4 |
| 10.0 | 8.9 | 9.9 |
| 17.7 | 10.0 | 12.5 |
| 18.3 | 15.9 | 13.9 |
| 19.5 | 18,.1 | 16.9 |
| 20.1 | 18.9 | 18.2 |
| 21.0 | 20.0 | 18.9 |
| 22.9 | 22.0 | 20.0 |
| 28.4 | 24.2 | 20.7 |
| | 26.9 | 23.5 |

In the next step, the methyl ester moiety of compounds of general formula (Vb; Z═CO₂Me)-(Vk; Z═CO₂Me) is hydrolyzed to yield the free acids of general formula (Vb; Z═CO₂H)-(Vk; Z═CO₂H). The hydrolysis is carried out in the presence of a base, such as metal hydroxide, such as e.g. LiOH, NaOH, KOH, CsOH, Ca(OH)₂; quaternary ammonium hydroxide, e.g. benzyltrimethylammonium hydroxide; metal alkoxide, e.g. t-BuOK; metal carbonate, e.g. K₂CO₃, etc. Preferably KOH is used. As solvents those with a low water content are preferably used, but not limited to, solvents such as THF, MeOH, EtOH, t-BuOH and mixtures thereof. The preferred solvents are THF and t-BuOH or any mixture thereof.

The obtained compounds of general formula (Vb; Z═CO₂H)-(Vk; Z═CO₂H) are activated by reacting them with oxalyl chloride to yield compounds of general formula (Vb; Z═COCl)-(Vk; Z═COCl).

Compounds of general formula (Vb; Z═COCl)-(Vk; Z═COCl) are then coupled with the in situ generated 4-fluorophenylzinc chloride to yield compounds of general formula (IIb)-(IIk).

The compounds of general formula (Vb; Z═CON(Me)OMe)-(Vk; Z═CON(Me)OMe) can be prepared in one of the following ways (Scheme 2 and 3): Reaction of compounds of general formula (Vb; Z═COCl)-(Vk; Z═COCl) (Scheme 2) with N,O-dimethylhydroxylamine salt in the presence of a base such as any organic tertiary non-nucleofilic base such as for example triethylamine, N-ethyldiisopropylamine, or similar. Preferably N-ethyldiisopropylamine can be used. As solvent inert organic solvent may be used, but not limited to, solvents such as THF, dichloromethane and any mixture thereof. The preferred solvent is THF.

b) Reaction of compounds of general formula (Vb; Z═CO₂H)-(Vk; Z═CO₂H) (Scheme 2) with an acid activator in a solvent and subsequent reaction with N,O-dimethylhydroxylamine salt, in the presence of a suitable base. The suitable solvents can be selected from the group consisting of water, tetrahydrofuran, methanol, ethanol, acetonitrile, i-propanol, n-butanol, dichloromethane and N,N-dimethylformamide preferably methanol and acetonitrile. The reaction temperature is below the boiling temperature of the solvent used, preferably between about −10° C. to about 35° C. The activators for acid of general formula (Vb; Z═CO₂H)-(Vk; Z═CO₂H) can be 2-chloro- or 2-bromo-1-methylpyridinium iodide, [bis(2-methoxyethyl)amino]sulfur trifluoride, S-(1-oxido-2-pyridinyl)-1,3-dimethylpropyleneuronium tetrafluoroborate, S-(1-oxido-2-pyridinyl)-1,1,3,3-tetramethyluronium hexafluorophosphate, 2-chloro-4,6-dimethoxy-[1,3,5]-triazine, or 4-(4,6-dimethoxy-1,3,5-triazin-2-yl)-4-methylmorpholinium chloride, preferably 4-(4,6-dimethoxy-1,3,5-triazin-2-yl)-4-methylmorpholinium chloride. These activators are usually used in excess of 1 to 1.5 moles, preferably 1.1 to 1.3 moles per mole of the compounds of general formula (Vb; Z═CO₂H)-(Vk; Z═CO₂H). Bases used can be organic tertiary non-nucleophilic amines such as for example triethylamine, diethylpropylamine, diisopropylethylamine, N-methylpyrrolidine and N-methylmorpholine, preferably N-methylmorpholine, N-methylpiperidine, more preferably N-methylmorpholine. The bases can be in about 1 to 5 moles excess; preferably in 1.8 to 2.2 moles excess. N,O-dimethylhydroxylamine salt can be used in excess of 1 to 2 moles, preferably 1.3 to 1.6 moles per mole of compounds of general formula (Vb; Z═CO₂H)-(Vk; Z═CO₂H). c) Reaction of N,O-dimethylhydroxylamine salt with suitable organometalic reagent and subsequent reaction with compounds of general formula (Vb; Z═CO₂Me)-(Vk; Z═CO₂Me) (Scheme 3) in a suitable solvent. Organometalic reagents can be selected from the group consisting of trimethylaluminium, triethylaluminium, dimethylaluminium chloride, diethylaluminium chloride, isopropylmagnesium chloride and n-butyl-lithium. The suitable solvents may be dichloromethane, tetrahydrofuran, 2-methyltetrahydrofuran, toluene and N,N-dimethylformamide. The reaction temperature may be between −50° C. and 200° C., preferably between about 50° C. to 120° C.

Reaction of compounds of general formula (Vb; Z═COCl)-(Vk; Z═COCl) with benzotriazole affords compounds of general formula (Vb; Z═CO-benzotriazol-1-yl)-(Vk; CO-benzotriazol-1-yl) (Scheme 3). The inert solvent may be selected from the group consisting of tetrahydrofuran, 2-methyltetrahydrofuran, diglyme, dioxane, diethyl ether, diisopropyl ether, tert.-butyl methyl ether, cyclopentyl methyl ether, dichloromethane and toluene, preferably tetrahydrofuran and dichloromethane. Optionally the base can be added to the reaction mixture. The base may be selected from the group consisting of any organic tertiary non-nucleofilic base such as for example triethylamine, N-ethyldiisopropylamine, or similar. Preferably N-ethyldiisopropylamine can be used. The reaction temperature is below the boiling temperature of the solvent used, preferably between −78° C. to boiling temperature of the solvent, more preferably between −10° C. to 35° C.

The compounds of general formula (Vb; Z═CON(Me)OMe)-(Vk; Z═CON(Me)OMe) or (Vb; Z═CO-benzotriazol-1-yl)-(Vk; CO-benzotriazol-1-yl) can be coupled with corresponding organometalic reagent to provide compounds of general formula (IIb)-(IIk) in a solvent (Scheme 4). Organometalic reagents may be selected from the group consisting of 4-fluorophenylmagnesium bromide, 4-fluorophenyl-lithium, 4-fluorophenylcalcium bromide and 4-fluorophenylbarium bromide, preferably 4-fluorophenylmagnesium bromide and 4-fluorophenyl-lithium. Organometalic reagent can be used in excess of 1 to 5 moles, preferably 1.5 to 3 moles per mole of compound of general formula (Vb; Z═CON(Me)OMe)-(Vk; Z═CON(Me)OMe) or (Vb; Z═CO-benzotriazol-1-yl)-(Vk; CO-benzotriazol-1-yl). The inert solvent may be selected from the group consisting of tetrahydrofuran, 2-methyltetrahydrofuran, diglyme, dioxane, diethyl ether, diisopropyl ether, tert-butyl methyl ether, cyclopentyl methyl ether and toluene, preferably tetrahydrofuran and toluene. The reaction temperature is below the boiling temperature of the solvent used, preferably between −78° C. to boiling temperature of the solvent, more preferably between −78° C. to 35° C., for about 0.5 to 4 hours, preferably 1 hour. After completion of the reaction, the reaction mixture is acidified and extracted with suitable solvent.

Compounds of general formula (IIb)-(IIk) (scheme 5) can be prepared by reacting the compound of general formula (Vb; Z═COCl)-(Vk; Z═COCl) with 4-fluorophenylmagnesium bromide with a tridentate ligand in an inert solvent. Suitable tridentate ligands may be selected from the group consisting of N-methylmorpholine, N,N,N′,N′-tetramethylethylenediamine, N,N,N′,N′,N′-petramethyldiethylenetriamine and bis[2-(N,N-dimethylamino)ethyl]ether. Preferably bis[2-(N,N-dimethylamino)ethyl]ether can be used. An inert solvent may be selected from the group consisting of tetrahydrofuran, 2-methyltetrahydrofuran, diglyme, dioxane, diethyl ether, diisopropyl ether, tert-butyl methyl ether, cyclopentyl methyl ether or toluene, preferably tetrahydrofuran or toluene. The reaction temperature is below the boiling temperature of the solvent used, preferably between −78° C. to boiling temperature of the solvent, more preferably between −78° C. to 35° C.

Compounds of general formula (IIb)-(IIk) (scheme 5) can be prepared by reacting the compound of general formula (Vb; Z═COCl)-(Vk; Z═COCl) with 4-fluorophenylboronic acid in the presence of a base and a metal catalyst in a solvent. The coupling solvents for the reaction can be selected from a variety of known process solvents. Illustrative of the coupling solvents that can be utilized either singly or in combinations may be selected from the group consisting of benzene, toluene, tetrahydrofuran, dioxane, acetonitrile, acetone, N,N-dimethylformamide, N,N-dimethylacetamide, ethanol, methanol, propanol, water, 2-methyltetrahydrofuran, diethoxymethane, N-methylpyrrolidinone, hexamethylphosphoramide, supercritical CO₂ or any ionic liquids. The metal catalyst may be a complex of nickel, palladium, or platinum, preferably a palladium complex such as tetrakis[tri(4-methylphenyl)phosphine]palladium, tetrakis(triphenylphosphine)palladium, bis(dibenzylideneacetone)palladium, tris(dibenzylideneacetone)dipalladium, a phosphinated palladium II complex selected from the group consisting of: bis(triphenylphosphine)palladium chloride, bis(triphenylphosphine)-palladium bromide, bis(triphenylphosphine)palladium acetate, bis(triisopropylphosphite)-palladium chloride, bis(triisopropylphosphite)palladium bromide, bis-(triisopropylphosphite)palladium acetate, [1,2-bis(diphenylphosphino)ethane]palladium chloride, [1,2-bis(diphenylphosphino)ethane]palladium bromide, (1,2-bis(diphenylphosphino)ethane]-palladium acetate, 3-bis(diphenylphosphino)propane]palladium chloride, (1,3-bis(diphenylphosphino)propane]palladium bromide, (1,3-bis(diphenylphosphino)propane]-palladium acetate, [1,4-bis(diphenylphosphino)butane]palladium chloride, [1,4-bis-(diphenylphosphino)butane]palladium bromide, [1,4-bis(diphenylphosphino) butane]palladium acetate, palladium(II) chloride or palladium(II) acetate. Variety of bases can be used in the reaction, illustrative examples may be selected from the group consisting of organic tertiary non-nucleophilic bases such as for example triethylamine or diisopropylethylamine, inorganic bases such as for example potassium carbonate, sodium carbonate, sodium hydrogencarbonate, caesium carbonate, thallium carbonate, potassium hydroxide, sodium hydroxide, thallium hydroxide, or the alkoxides of these alkali metals. When an inorganic base insoluble in the organic solvent is used, dissolution in water may be necessary; the use of a phase-transfer catalyst such as tetra-n-butylammonium bromide or crown ether also facilitate the reaction. Organic solvent soluble bases such as tetra-n-butylammonium carbonate or tetra-n-butylammonium hydroxide, benzyltrimethylammonium carbonate, benzyltrimethylammonium methyl carbonate, benzyltrimethylammonium methoxide or benzyltrimethylammonium hydroxide, or other basic tetraalkylammonium compounds can be used as well.

Compounds of general formula (IIa)-(IIk) thus obtained as disclosed in Schemes 1-5.

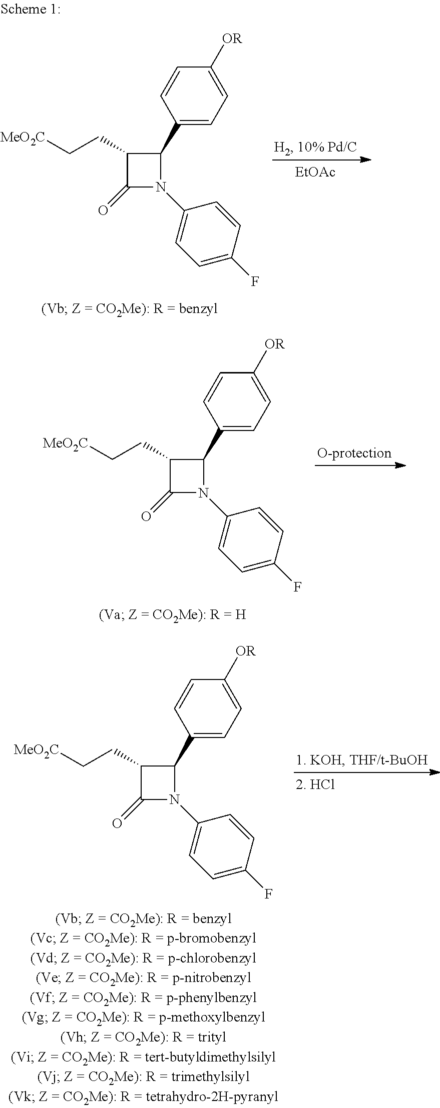

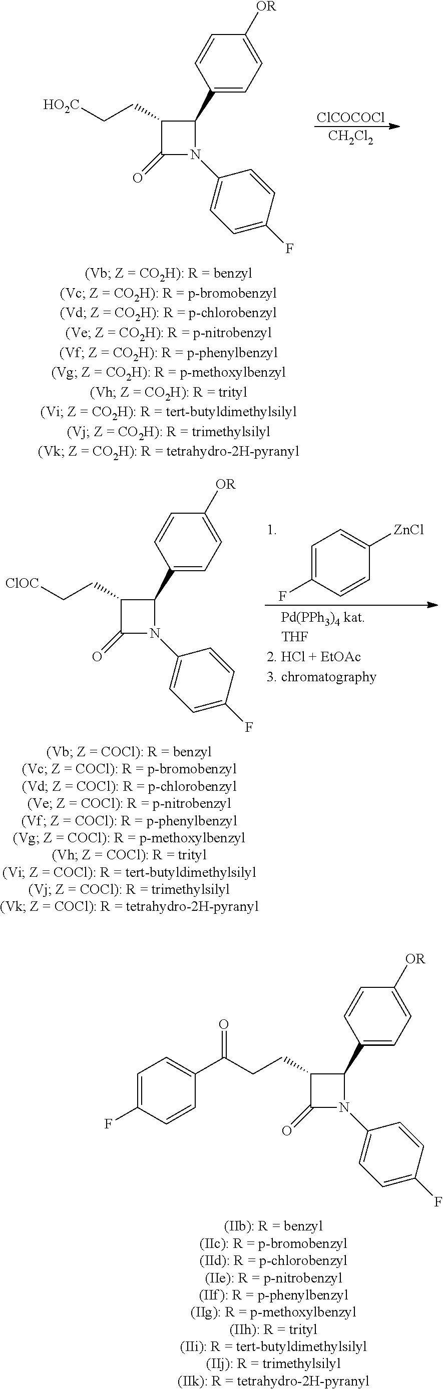

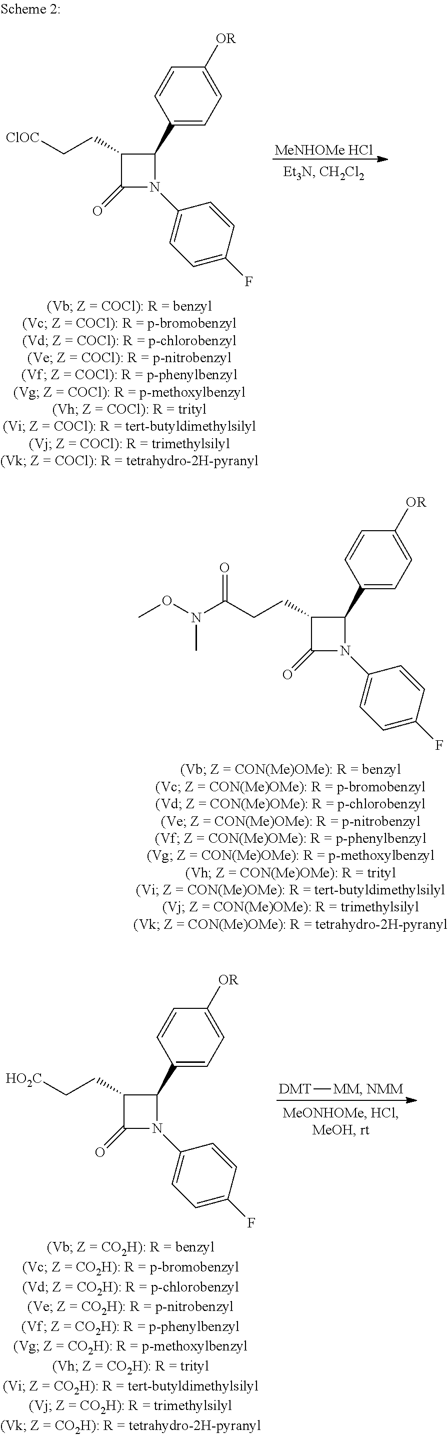

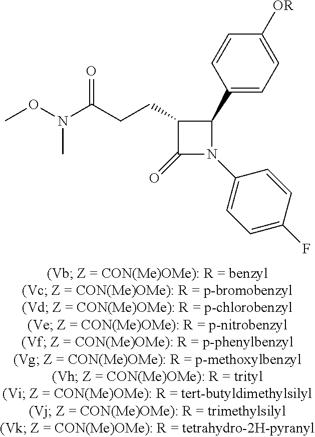

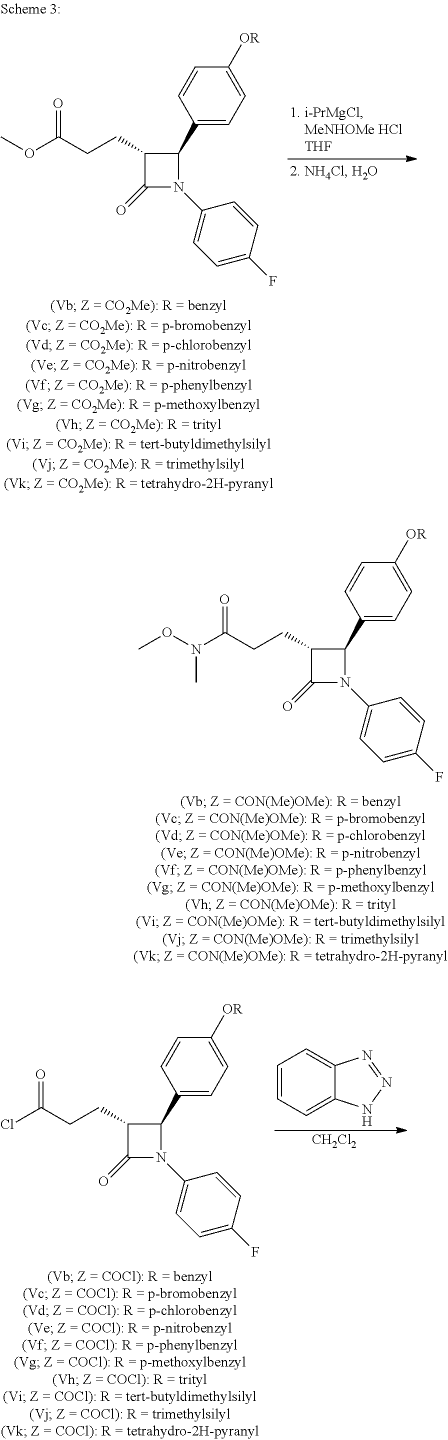

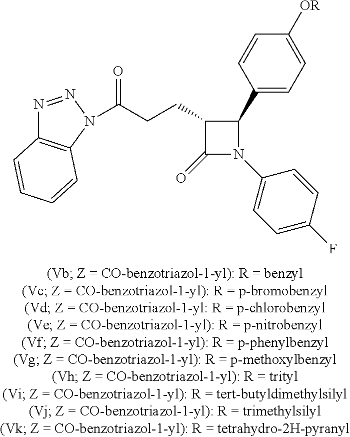

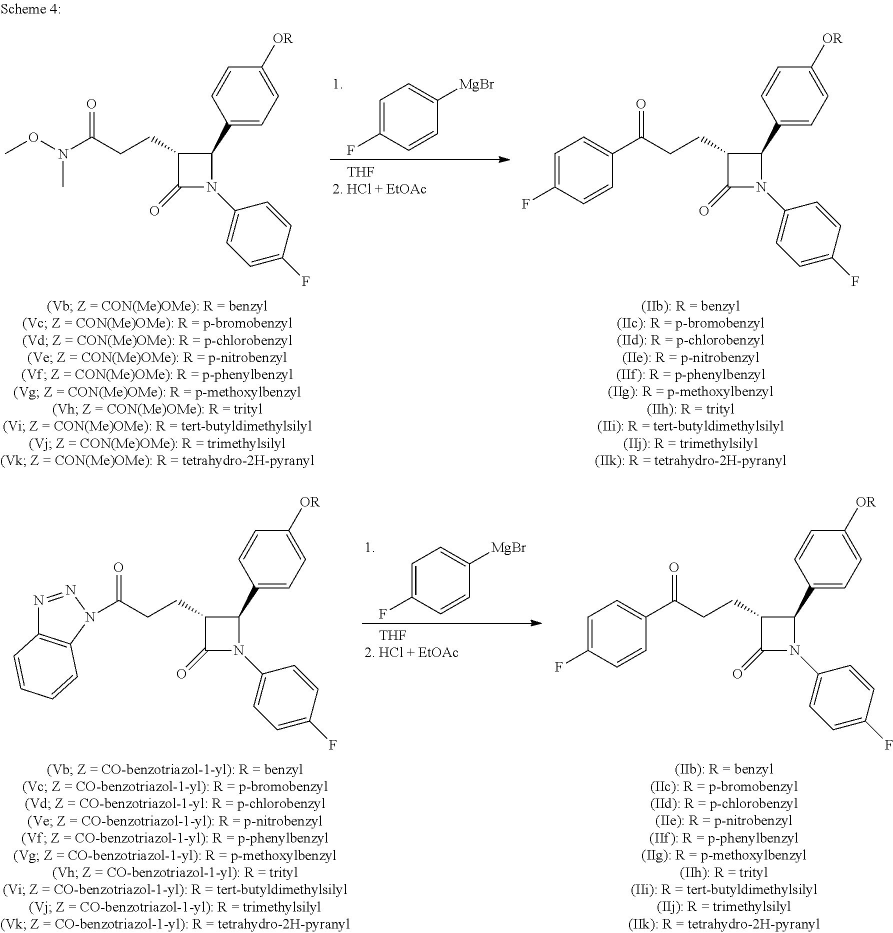

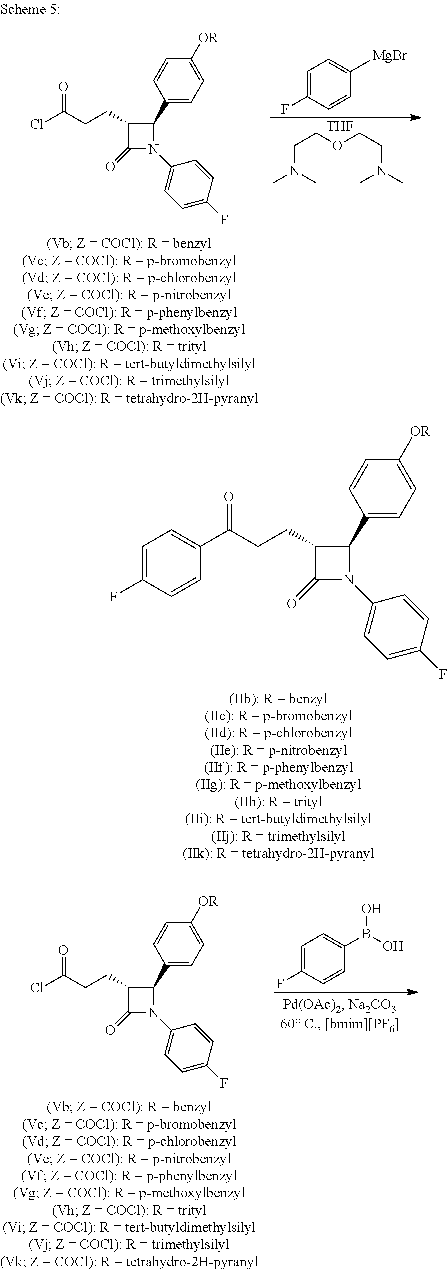

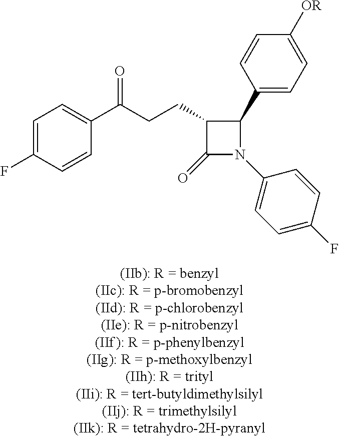

Said compounds are further reduced according to the present invention as disclosed in Scheme 6 yielding compounds of general formula (Ib)-(Ik).

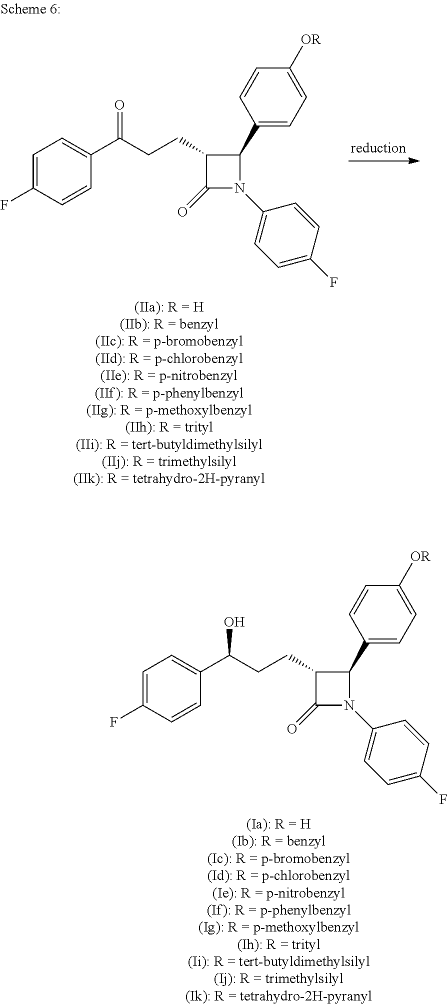

Deprotection of the group R of compounds of general formula (Ib)-(Ik) can be provided by any process known in the art, so that ezetimibe can be obtained. In the preferred case, hydrogenation of compounds of general formula (Ib)-(Ih) in the presence of Pd/C or treatment of the compounds of general formula (Ih)-(Ik) with acidic reagents is used in the R group de-protection step.

In yet another embodiment of the present invention, novel intermediates represented by the general formula (V)

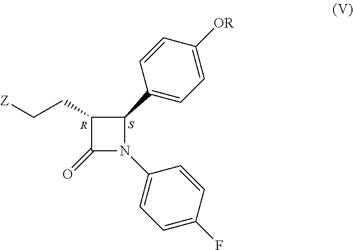

wherein:
Z represents COCl, COOH, COOMe, CON(Me)OMe, CON(Me)OEt, or CO-benzotriazol-1-yl;
R represents a protective group as described previously for ketone (II). The R protecting group can be introduced by known methods as for example described by T. W. Greene and P. G. M. Wuts in “Protective Groups in Organic Synthesis”, 1999, John Wiley & Sons. were prepared. The compounds are useful to easily access p-fluoroacetophenones of general formula (II).

Examples for the compounds of the general formula (V) are:

### Methyl 3-[(2S,3R)-1-(4-fluorophenyl)-2-(4-hydroxyphenyl)-4-oxoazetidinyl]propionate

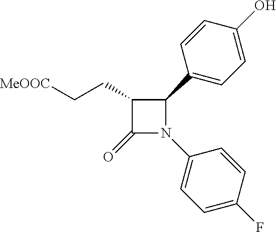 [SMILES: COC(=O)CC[C@H]1C(=O)N(c2ccc(F)cc2)[C@@H]1c1ccc(O)cc1] [0080] (Va; Z═CO₂Me, R═H)

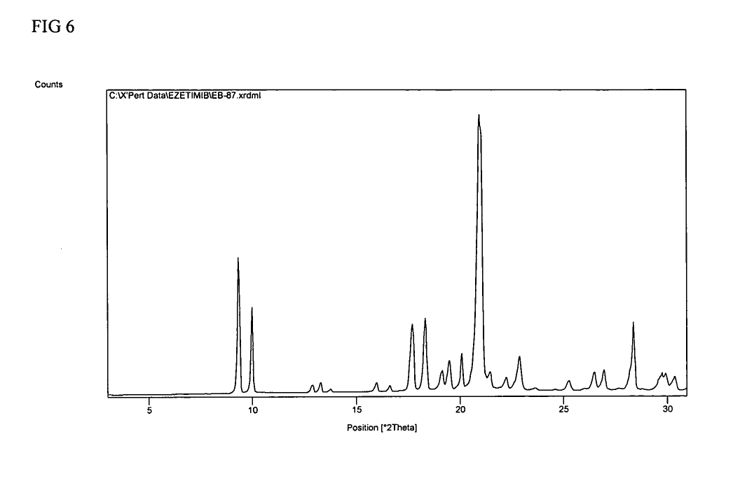

The powder X-ray diffraction pattern is illustrated in FIG. 6.

### Methyl 3-{(3R,4S)-1-(4-fluorophenyl)-2-oxo-4-[4-(trityloxy)phenyl]azetidin-3-yl}propionate

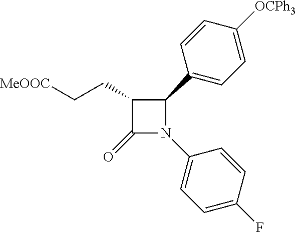 [0083] (Vh; Z═CO₂Me, R═CPh₃)

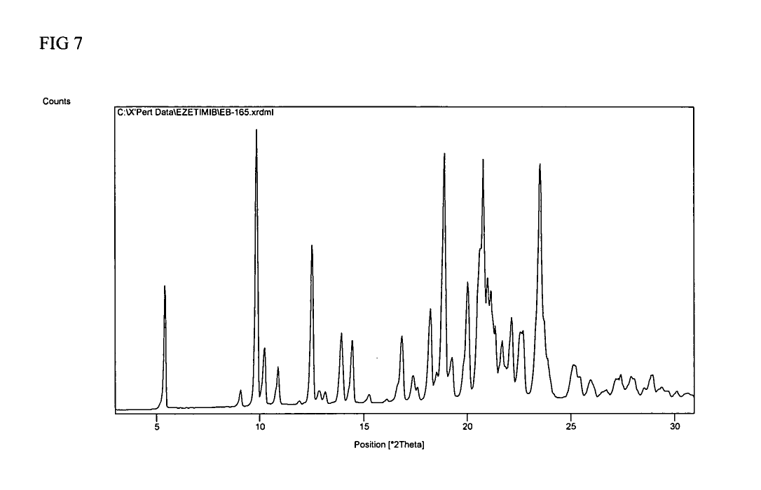

The powder X-ray diffraction pattern is illustrated in FIG. 7.

According to still another embodiment, a compound of formula

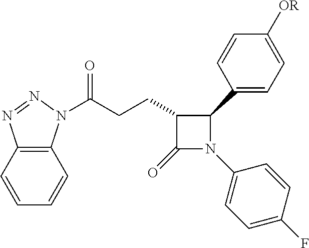

wherein R is selected from the group consisting of p-bromobenzyl, p-chlorobenzyl, p-nitrobenzyl, p-methoxybenzyl, trityl, tert-butyldimethylsilyl, benzyl, p-phenylbenzyl, trimethylsilyl and tetrahydro-2H-pyranyl is provided. Preferably R is benzyl.

The present compounds were characterized with regard to their melting points (T m) by means of Koffler melting point apparatus with accuracy of approximately ±1° C. and X-ray powder diffraction patterns (obtained by a Phillips PW3040/60 X'Pert PRO diffractometer using CuKα radiation of 1,541874 Å). Images of particles were taken on a Microscope Olympus BX 50 equipped with Olympus camera DP70.

Ezetimibe prepared according to the present invention have a purity of at least about 90%, more preferably at least about 95% and most preferably at least about 99% as measured by HPLC.

Ezetimibe prepared according to the process of the present invention can be isolated/crystallized or further purified by processes known from the prior art (as for example WO 2004/099132, WO 2005/066120, WO 2006/060808, WO 2005/062897, WO 2005/009955, WO 2006/050634, IPCOM000131677, G.Y.S.K.Swamy at all, Acta Cryst. (2005). E61, 03608-03610). Solvents and/or reagents that could be used are n-butanol, n-propanol, chloroform, THF, acetone, bistrimethyl silyl acetamide, diethyl ketone, ethyl acetate, methanol and the like, particularly as for example isopropanol/water, methanol/water, ethanol/water etc.

Anhydrous form A (marked as anhydro form A) characterized by powder X-ray diffraction peaks at 8.3; 13.9; 16.4; 18.7; 19.0; 20.1; 23.6; 23.9; 25.6; 29.7° 2Θ, is obtained when the crude ezetimibe is dissolved in an anhydrous solvent. Anhydro form A can be characterized by the water content of less than about 0.5%, preferably less than about 0.3% as determined by Karl Fischer analysis. Anhydro form A can be obtained by exposing the hydrated form H to relative humidity lower than about 20% for about 12 h, by drying of the hydrated form H in an air dryer at a relative humidity of less than about 50%, preferably less than about 40% and a temperature of about 30 to about 70° C., preferably at about 40 to 50° C., by vacuum drying at ambient temperature and pressure of less than about 100 mbar, or by crystallization of anhydro form A or hydrated form H from tert-butyl methyl ether/n-heptane.

Hydrated form of ezetimibe (marked as hydrated form H) characterized by powder X-ray diffraction peaks at 7.9; 15.8; 18.6; 19.3; 20.7; 21.7; 22.9; 23.4; 24.5; 25.2° 2Θ, is obtained when the crude ezetimibe is dissolved in a water containing solvent. It is further characterized by the water content of from about 4 to about 6%, preferably from about 4 to about 4.5% as determined by Karl Fischer analysis.

The crystals of ezetimibe anhydro form A or hydrated form H can have a particle size of less than about 100 microns, preferably less than 50 microns, more preferably less than about 30 microns. Crystals of ezetimibe anhydro form A or hydrated form H may be further micronized by milling or any other process known from the prior art to obtain the micronized crystals of particle size of less than about 30 microns, preferably less than about 20 microns and more preferably less than about 10 microns.

Ezetimibe anhydro form A or hydrated form H is substantially free of other polymorphic forms, preferably it contains not more than 20%, more preferably not more than 10%, most preferably not more than 5% of other polymorphic forms.

While investigating the solubility of anhydro form A and hydrated form H in different solvents it was surprisingly found out that by using tert-butanol, known as anhydrous compound, as a solvent in the crystallization step a new form (named as form S) is obtained.

According to a preferred embodiment, ezetimibe form S specified by an X-ray powder diffraction pattern exhibiting peaks at the following diffraction angles: 7.3, 15.3, 16.7, 18.7, 21.8, 24.0° 2Θ is provided.

According to an embodiment, ezetimibe form S is specified by an X-ray powder diffraction pattern exhibiting additional peaks at the following diffraction angles: 6.2, 20.1, 25.3° 2Θ.

Another embodiment of the present invention is form S of ezetimibe characterized by powder X-ray diffraction peaks at the following diffraction angles

| No. | Pos. [°2Θ.] | d-spacing [A] | Rel. Int. [%] |
|---|---|---|---|
| 1 | 6.2 | 14.21 | 11 |
| 2 | 7.3 | 12.15 | 23 |
| 3 | 15.3 | 5.78 | 32 |
| 4 | 16.7 | 5.31 | 63 |
| 5 | 18.7 | 4.75 | 100 |
| 6 | 20.1 | 4.41 | 80 |
| 7 | 21.8 | 4.08 | 57 |
| 8 | 24.0 | 3.71 | 51 |
| 9 | 25.3 | 3.52 | 37 |

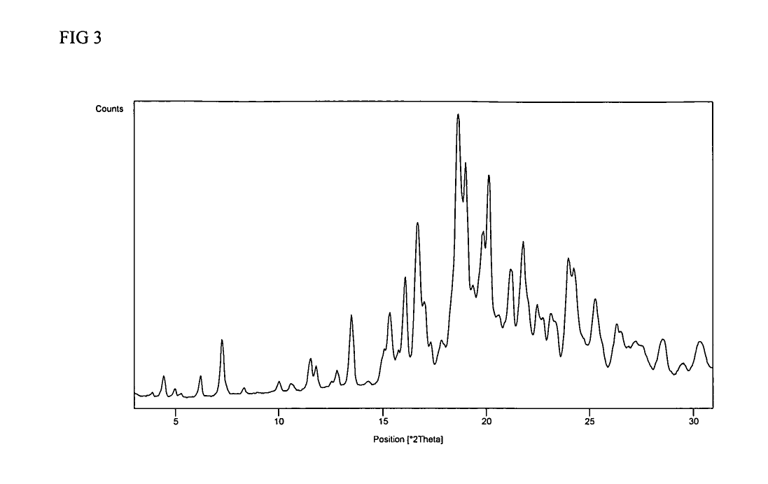

According to an embodiment, ezetimibe form S has an X-ray powder diffraction pattern as shown in FIG. 3.

Ezetimibe form S can be characterized by ¹H-NMR peaks at δ=1.11 (s, t-Bu), 1.6-1.9 (m, 4H, H-1′, H-2′), 3.08 (m, 1H, H-3), 4.20 (s, t-Bu-OH), 4.49 (m, 1H, H-3′), 4.80 (d, J=2.3 Hz, 1H, H-4), 5.29 (br d, J=2.7 Hz, 1H, OH-3′), 6.73-6.78 (m, 2H, Ar—H), 7.08-7.34 (m, 10H, Ar—H), 9.54 (br s, 1H, Ar—OH). NMR spectra were measured on a Varian Inova 300 MHz spectrometer in DMSO-d₆.

Ezetimibe form S can be further characterized by solid-state ¹³C-NMR. Solid-state NMR ¹³C spectroscopy can be carried out using ¹³C cross polarization/magic angle spinning

(CP/MAS). Solid state analysis was performed using a Varian Inova 600 MHz spectrometer operating at a carbon frequency 150.830 MHz, equipped with a complete solids accessory and Varian 3.2 mm NB Double Resonance HX MAS Solids Probe. Data were recorded at ambient temperature and 10 kHz spinning frequency, with 5.0 ms contact time and a repetition time of 2.0 s.

Chemical shifts were referenced externally to the methyl group of hexamethylbenzene (δ=17.3 ppm) by sample replacement and were observed at: 28.4, 31.3, 37.5, 39.4, 60.3, 64.1, 64.7, 70.7, 73.9, 74.7, 75.3, 78.1, 115.3, 117.5, 119.9, 125.9, 127.0, 128.8, 129.9, 130.6, 131.8, 135.0, 135.8, 140.1, 140.7, 142.4, 143.8, 156.0, 157.9, 159.9, 160.8, 161.6, 162.3, 167.1, 168.2, 170.2.

From the solid state NMR analysis we can conclude that form A and form H contains only one molecule in the crystallographic asymmetric unit whereas form S contains at least two molecules in the asymmetric unit.

Yet another embodiment of the present invention is the process for the preparation of ezetimibe form S by dissolving anhydro form A and/or hydrated form H and/or any other polymorphic form in tert.-butanol. The resulting solution is cooled to room temperature, the precipitated material is filtered and dried. In case that no precipitation takes place, the crystallization occurs after seeding with crystals of ezetimibe form S. The obtained ezetimibe form S has purity more than 90%, preferably more than 99%, more preferably more than 99.6%.

According to an embodiment ezetimibe form S has a purity of more than 90%, preferably more than 99%, more preferably more than 99.6%.

According to an embodiment ezetimibe form S contains from about 8 to about 15% of tert.-butanol, preferably from about 10 to about 12% of tert.-butanol. Ezetimibe form S may be further dried-desolvated in order to be appropriate for incorporation into the pharmaceutical composition.

According to another embodiment, ezetimibe form S is characterized by a water content of about 0 to about 2%, determined by Karl Fischer analysis, a method well known to the skilled person. Preferably, the water content is about 0.5 to about 1.5% as determined by Karl Fischer analysis.

According to another embodiment, the crystals of said ezetimibe form S have a particle size of less than about 100 microns, preferably less than 50 microns, more preferably less than about 30 microns.

Crystals of ezetimibe form S may be further micronized by milling or any other process known from the prior art to obtain the micronized crystals of particle size of less than about 30 microns, preferably less than about 20 microns and more preferably less than about 10 microns. According to still another embodiment, the micronized crystals of said ezetimibe form S have a particle size of less than about 30 microns, preferably less than 20 microns, more preferably less than about 10 microns.

According to an embodiment, ezetimibe form S contains not more than 20%, preferably not more than 10%, more preferably not more than 5% and most preferably not more than 1% of other polymorphic forms.

Crystals of ezetimibe form S are in the form of small particles and bigger agglomerates of irregular shape.

Surprisingly ezetimibe form S is stable upon drying, even at temperature of about 70° C. only minimal loss of drying was observed in comparison to hydrated form H which is converted to anhydrous form A already at temperature of about 40° C. The stability to heating is very important factor in the preparation and storage of pharmaceutical compositions.

In addition to extraordinary thermal stability of ezetimibe form S, we surprisingly found out, that solvate can be efficiently and completely de-solvated if suspended in appropriate anti-solvent or any mixtures thereof or solvent/anti-solvent system and stirred at defined temperatures during defined time. Solvents and anti-solvents, that can be used in process can be selected from the group of cyclic and linear C₅-C₆ hydrocarbons, ethers, esters, lover ketons, alcohols, toluene, acetonitrile, halogenated lower hydrocarbons, water and mixture thereof, more preferably toluene, water, acetone, isopropanol and mixture thereof, most preferably water.

Concentration of ezetimibe can range from 0.01 g/mL to 1 g/mL, preferably 0.02 g/mL to 0.2 g/mL. Temperature during de-solvatation can be controlled between 4° C. to 95° C., preferably 20 to 60° C. Stirring of suspension is performed from 1 minute to 3 hours, more preferably from 5 minutes to 1 hour. With appropriate control of process parameters and solvent mixtures during the de-solvatation step primary particles of average size between 1 and 50 μm can be prepared, preferably between 1 μm and 15 μm, that are substantially free of agglomerates. It is well known, that small primary particles are deciding factor in improving bioavailability of water insoluble drugs. With said process of de-solvatation of ezetimibe form S, ezetimibe for direct use in pharmaceutical composition with good bioavailability is prepared.

According to a preferred embodiment a pharmaceutical composition comprising a therapeutically effective amount of ezetimibe in any polymorphic form is provided, which is prepared according to the present invention and optionally mixed with one or more active substances, and one or more pharmaceutically acceptable ingredients.

According to an embodiment, the pharmaceutical composition comprises a therapeutically effective amount of ezetimibe form S optionally mixed with one or more active substances, and one or more pharmaceutically acceptable ingredients.

According to another embodiment, the use of a therapeutically effective amount of ezetimibe is provided, wherein ezetimibe is prepared according to invention and is suitable for lowering cholesterol levels in a mammal in need of such treatment.

According to an embodiment, the use of a therapeutically effective amount of ezetimibe form S is provided for lowering cholesterol levels in mammal in need of such treatment.

In another embodiment the present invention provides a pharmaceutical composition containing ezetimibe prepared according to the process of the present invention and being in any known polymorphic form as for example anhydro form A, hydrated form H or form S, optionally mixed with other active ingredients such as for example HMG-CoA reductase inhibitors and at least one pharmaceutical acceptable excipient.

The pharmaceutical composition according to the present invention can be in any conventional form, preferably an oral dosage form such as a capsule, tablet, pill, liquid, emulsion, granule, suppositories, powder, sachet, suspension, solution, injection preparation and the like. The formulations/compositions can be prepared using conventional pharmaceutically acceptable excipients. Such pharmaceutically available excipients and additives include fillers/diluents, binders, disintegrants, glidants, lubricants, wetting agents, preservatives, stabilizers, antioxidants, flavouring agents, coloring agents, emulsifier. Preferably, the oral dosage form is a tablet.

Suitable diluents include lactose, calcium carbonate, dibasic calcium phosphate anhydrous, dibasic calcium phosphate dehydrate (for example Emcompress®), tribasic calcium phosphate, microcrystalline cellulose (such as for example Avicel® PH 101, Avicel® PH 102 etc), powdered cellulose, silicified microcrystalline cellulose (for example Prosolv®), dextrates (for example Emdex®), dextrose, fructose, glucose, lactitol, lactose anhydrous, lactose monohydrate, spray-dried lactose, magnesium oxide, magnesium carbonate, maltitol, maltodextrin, maltose, mannitol, starch, sucralose, sucrose, xylitol and others. Also, special excipients for direct compression such as cellactose or starlac can be used. Preferably, lactose monohydrate, mannitol and microcrystalline cellulose are used. The diluent can be present in an amount between 30 and 90 w %, preferably between 40 and 80 w %.

Binders are selected from the group consisting of gelatin, guar gum, cellulose derivatives (hydroxyethyl cellulose HEC, Hydroxyethylmethyl cellulose HEMC, hydroxypropyl cellulose HPC (for example Klucel® EF or Klucel® LF), hydroxypropylmethyl cellulose HPMC (Pharmacoat® 603 or 606), methyl cellulose MC . . . ), polymetacrylates, polyvinyl alcohol, povidone (of different grades, for example povidone K12, K15, K17, K25, K30 etc), starch and its derivatives (hydroxyethyl starch, pregelatinized starch) etc. The binder can be present in an amount between 1 and 10 w %, preferably between 2 and 8 w %.

Suitable disintegrants include, but are not limited to, carboxymethyl cellulose sodium, carboxymethyl cellulose calcium, croscarmellose sodium, crospovidone, starch and modified starches (sodium starch glycolate—Primojel®), low substituted hydroxypropyl cellulose, magnesium aluminium silicate, calcium silicate and others and any mixtures thereof. Preferably, low substituted hydroxypropyl cellulose is used. The disintegrant can be present in an amount between 1 and 50 w %, preferably between 2 and 40 w % and more preferably between 4 and 30 w %.

Suitable surface active agents (solubilising agents) include, but are not limited to sodium laurylsulfate, glyceryl esters, polyoxyethylene glycol esters, polyoxyethylene glycol ethers, polyoxyethylene sorbitan fatty acid esters, sulphate containing surfactants, or polyoxyethylene/polyoxypropylene copolymers. The most preferred is sodium laurylsulfate. The surface active agent can be present in an amount between 0 and 5 w %, preferably between 0.5 and 3 w %.

Possible antioxidants include, but are not limited to, vitamin E acetate, α-tocopherol, ascorbyl palmitate, butylated hydroxyanisole (BHA), butylated hydroxytoluene (BHT), propyl gallate, citric acid, dithiotreitol, or tocopherol polyethyleneglycol succinate (TPGS). Chelating agents can also be used as antioxidants, for example EDTA or cyclodextrins.

Suitable glidants are silicon dioxide, talc and aluminium silicate.

Lubricants are preferably selected from the group consisting of magnesium stearate, sodium stearyl fumarate, sucrose esters of fatty acids, stearic acids and the like.

Sweeteners can be selected from aspartame, saccharin sodium, dipotassium glycirrhizinate, aspartame, thaumatin and the like.

The pharmaceutical composition according to the present invention may be prepared by well known technological processes such as direct compression or wet granulation, dry granulation or lyophilization. Preferably, wet granulation process in fluid bed system is used.

The ezetimibe prepared according to the present invention can be formulated in the pharmaceutical composition as described in WO 2007/003365.

The present invention is illustrated by the following examples without limiting it thereto.

### EXAMPLES

### Reference Example (EP 0720599, Example 6)

### Synthesis of ezetimibe from methyl 3-{(2S,3R)-2-[4-(benzyloxy)phenyl]-1-(4-fluorophenyl)-4-oxoazetidin-3-yl}propionate (Vb; Z═CO₂Me)

### Procedure 1:

To a solution of methyl 3-{(2S,3R)-2-[4-(benzyloxy)phenyl]-1-(4-fluorophenyl)-4-oxoazetidin-3-yl}propionate (Vb; Z═CO₂Me) (1.6 g, 3.7 mmol) in methanol (3.5 ml) and water (1.5 ml) was added lithium hydroxide monohydrate (155 mg, 3.7 mmol). The mixture was stirred at room temperature for 1.5 h, then additional amount of lithium hydroxide monohydrate (54 mg, 1.3 mmol) was added and stirring continued for 3 h. 1M hydrochloric acid (5 ml) and ethyl acetate (15 ml) were added, the organic layer was washed 3 times with water and dried over sodium sulfate. Concentration in vacuo afforded the acid (Vb; Z═CO₂H) (1.4 g, 89%) as an amber colored foam.

### Procedure 2:

To a solution of methyl 3-{(2S,3R)-2-[4-(benzyloxy)phenyl]-1-(4-fluorophenyl)-4-oxoazetidin-3-yl}propionate (Vb; Z═CO₂Me) (32 g, 74 mmol) in methanol (70 ml) and water (30 ml) was added lithium hydroxide monohydrate (3.1 g, 74 mmol). The mixture was stirred at room temperature for 1.5 h, then additional amount of lithium hydroxide monohydrate (1.08 g, 26 mmol) was added and stirring continued for 5.45 h. 1M hydrochloric acid (100 ml) and ethyl acetate (110 ml) were added, the organic layer was washed with water and dried over sodium sulfate. Concentration in vacuo afforded the acid (Vb; Z═CO₂H) (31.86 g, quant.) as an amber colored foam.

### Step 2

To a solution of 3-{(2S,3R)-2-[4-(benzyloxy)phenyl]-1-(4-fluorophenyl)-4-oxoazetidin-3-yl}propionic acid (Vb; Z═CO₂H) (30 g, 71.5 mmol) in dichloromethane (52 ml) was added 2M solution of oxalyl chloride in dichloromethane (53 ml, 106 mmol) and the mixture was stirred at room temperature for 16.5 h. Concentration in vacuo gave the acid chloride (Vb; Z═COCl) (31.45 g, quant.) as a viscous amber colored oil.

### Step 3

To a solution of dried zinc chloride (10.2 g, 73.4 mmol) in tetrahydrofuran (66 ml) was added dropwise 1M solution of 4-fluorophenylmagnesium bromide (73 ml) in tetrahydrofuran at 4° C. while stirring. Tetrakis(triphenylphosphine)palladium (4.12 g, 3.6 mmol) was added to the resulting suspension of 4-fluorophenylzinc chloride at 0° C., followed by a solution of 3-{(2S,3R)-2-[4-(benzyloxy)phenyl]-1-(4-fluorophenyl)-4-oxoazetidin-3-yl}propionyl chloride (Vb; Z═COCl) (31.45 g, 71.5 mmol) in tetrahydrofuran (69 ml) and the cooling bath was removed. After 4.5 h of stirring 1M hydrochloric acid (20.5 ml) and ethyl acetate (200 ml) were added, the organic layer was washed with water (100 ml) and dried over sodium sulfate. Concentration afforded an oil which was purified by repeated silica gel chromatography with toluene/isopropanol (100/1). (3R,4S)-4-[4-(benzyloxy)phenyl]-1-(4-fluorophenyl-3-[3-(4-fluorophenyl)-3-oxopropyl]azetidin-2-one (IIb) (13.2 g, 37%) was obtained as a brown colored oil.

### Step 4

A solution of (3R,4S)-4-[4-(benzyloxy)phenyl]-1-(4-fluorophenyl)-3-[3-(4-fluorophenyl)-3-oxopropyl]azetidin-2-one (IIb) (6.54 g, 13 mmol) and (R)-1-methyl-3,3-diphenyltetrahydro-1H,3H-pyrrolo[1,2-c][1,3,2]oxazaborole (2.9 ml, 1M in toluene) in tetrahydrofuran (20.6 ml) was cooled to −20° C., then borane-dimethylsulfide complex (2M in tetrahydrofuran; 5.85 ml, 11.7 mmol) was added dropwise over 1.5 h at −18° C. The stirring was continued for additional 1 h at −19° C., then methanol (3.5 ml) and 1M hydrochloric acid (27.5 ml) were carefully added. The mixture was extracted with ethyl acetate (41 ml), the organic layer was washed with water (2×48 ml) and dried over sodium sulfate. Concentration afforded crude (3R,4S)-4-[4-(benzyloxy)phenyl]-1-(4-fluorophenyl)-3-[(S)-3-(4-fluorophenyl)-3-hydroxypropyl]azetidin-2-one (Ib) (5.625 g, 66.3%) as a brown colored foam of chemical purity 77.1%.

### Step 5

To a solution of (3R,4S)-4-[4-(benzyloxy)phenyl]-1-(4-fluorophenyl)-3-[(S)-3-(4-fluorophenyl)-3-hydroxypropyl]azetidin-2-one (Ib) (0.828 g, 1.66 mmol) in absolute ethanol (5.4 mL) was added 10% palladium on carbon (62 mg, Heraeus). The reaction mixture was shaken in a pressure bottle under pressure of hydrogen gas (4 bar) for 40 h. Then additional amount of catalyst (62 mg) was added and the hydrogenolysis continued until reaction was estimated to be complete according to TLC analysis (toluene/ethyl acetate=9/1). The catalyst was removed by filtration and washed with absolute ethanol (40 ml). The resulting solution was concentrated in vacuo to give crude ezetimibe (0.615 g, 90.7%) as a brownish solid. Immediate XRPD analysis showed the sample to be a mixture of amorphous and crystalline phases (anhydro<hydrated). Reanalysis after 9 days revealed slightly lesser amount of amorphous phase and prevalance of the anhydro over hydrated form in the crystalline phase. 0.438 g of this material was purified by recrystallization from ethanol/water (5/1, 3.7 ml). After stirring at room temperature for cca. 80 min and cooling for 15 min in an ice bath the crystals were filtered and washed with cold ethanol/water (1/1) mixture (6 ml) to yield ezetimibe (0.276 g) in hydrated form H according to XRPD analysis, which had mp 159-161.5° C.

### Example 1

### 3-{(2S,3R)-2-[4-(benzyloxy)phenyl]-1-(4-fluorophenyl)-4-oxoazetidin-3-yl}propionic acid (Vb; Z═CO₂H)

Procedure 1: Hydrolysis with KOH in THF/t-BuOH=1/3, small scale

To a solution of methyl 3-{(2S,3R)-2-[4-(benzyloxy)phenyl]-1-(4-fluorophenyl)-4-oxoazetidin-3-yl}propionate (Vb; Z═CO₂Me) (1.6 g, 3.7 mmol) in tetrahydrofuran (1 ml) and tert-butanol (3 ml) was added powdered potassium hydroxide (244 mg, 3.7 mmol). The mixture was stirred at room temperature for 1 h, then additional amount of powdered potassium hydroxide (90 mg, 1.3 mmol) was added and stirring continued for 1 h. 1M hydrochloric acid (5 ml) and ethyl acetate (18 ml) were added, the organic layer was washed 3 times with water and dried over sodium sulfate. Concentration in vacuo afforded the acid (Vb; Z═CO₂H) (1.5 g, 95%) as a viscous oil.

Procedure 2: Hydrolysis with KOH in THF/t-BuOH=1/3, larger scale

To a solution of methyl 3-{(2S,3R)-2-[4-(benzyloxy)phenyl]-1-(4-fluorophenyl)-4-oxoazetidin-3-yl}propionate (Vb; Z═CO₂Me) (32 g, 74 mmol) in tetrahydrofuran (20 ml) and tert-butanol (60 ml) was added powdered potassium hydroxide (4.88 g, 74 mmol). The mixture was stirred at room temperature for 1.5 h. Then 1M hydrochloric acid (100 ml) and ethyl acetate (200 ml) were added. The organic layer was washed 3 times with water and dried over sodium sulfate. Concentration in vacuo afforded the acid (Vb; Z═CO₂H) (30.9 g, 99%) as an amber foam.

Procedure 3: Hydrolysis with t-BuOK+H₂O in THF

To a solution of methyl 3-{(2S,3R)-2-[4-(benzyloxy)phenyl]-1-(4-fluorophenyl)-4-oxoazetidin-3-yl}propionate (Vb; Z═CO₂Me) (100 mg, 0.231 mmol) in tetrahydrofuran (2 ml) and water (10 mg, 0.462 mmol) was added potassium tert-butoxide (0.54 g, 1.85 mmol). The resulting suspension was stirred at room temperature for 72 h, then 1M hydrochloric acid (2 ml) and diethyl ether (20 ml) were added. The organic layer was washed 3 times with water and dried over sodium sulfate. Concentration in vacuo afforded the acid (Vb; Z═CO₂H) (86 mg, 89%) as a viscous oil.

Procedure 4: Hydrolysis with t-BuOK in H₂O/THF mixture

To a solution of methyl 3-{(2S,3R)-2-[4-(benzyloxy)phenyl]-1-(4-fluorophenyl)-4-oxoazetidin-3-yl}propionate (Vb; Z═CO₂Me) (50 mg, 0.146 mmol) in tetrahydrofuran (4 ml) and water (53 mg, 2.92 mmol) was added potassium tert-butoxide (0.54 g, 1.85 mmol). The resulting suspension was stirred at room temperature for 1 h, then heated to 50° C. for additional 1.5 h. After cooling to room temperature, 0.5M hydrochloric acid (2 ml) and diethyl ether (10 ml) were added to the reaction mixture. The organic layer was dried over sodium sulfate and concentrated in vacuo to afford the acid (Vb; Z═CO₂H) (45 mg, 92%) as a viscous oil.

Procedure 5: Hydrolysis with KOH in THF

To a solution of methyl 3-{(2S,3R)-2-[4-(benzyloxy)phenyl]-1-(4-fluorophenyl)-4-oxoazetidin-3-yl}propionate (Vb; Z═CO₂Me) (50 mg, 0.146 mmol) in tert-butanol (1.5 ml) was added powdered potassium hydroxide (13 mg, 0.232 mmol). The mixture was stirred at room temperature for 2 h, then 0.5M hydrochloric acid (2 ml) and tert-butyl methyl ether (15 ml) were added. The organic layer was washed 3 times with water, dried over sodium sulfate and concentrated in vacuo to obtain the acid (Vb; Z═CO₂H) (45 mg, 92%) as a viscous oil.

### Example 2

### 3-{(2S,3R)-2-[4-(4-bromobenzyloxy)phenyl]-1-(4-fluorophenyl)-4-oxoazetidin-3-yl}propionic acid (Vc; Z═CO₂H)

To a solution of methyl 3-{(2S,3R)-2-[4-(4-bromobenzyloxy)phenyl]-1-(4-fluorophenyl)-4-oxoazetidin-3-yl}propionate (Vc; Z═CO₂Me) (11.5 g, 22.4 mmol) in tetrahydrofuran (27 ml) and tert-butanol (36 ml) was added powdered potassium hydroxide (1.75 g, 22.4 mmol). The mixture was stirred at room temperature for 0.5 h, then 1M hydrochloric acid (30 ml) and ethyl acetate (100 ml) were added. The organic layer was washed 3 times with water and dried over sodium sulfate. Concentration in vacuo afforded the acid (Vc; Z═CO₂H) (9.2 g, 82%) as an almost colorless foam.

### Example 3

### 3-{(2S,3R)-2-[4-(4-chlorobenzyloxy)phenyl]-1-(4-fluorophenyl)-4-oxoazetidinyl}propionic acid (Vd; Z═CO₂H)

To a solution of methyl 3-{(2S,3R)-2-[4-(4-chlorobenzyloxy)phenyl]-1-(4-fluorophenyl)-4-oxoazetidin-3-yl}propionate (Vd; Z═CO₂Me) (12.78 g, 27.3 mmol) in tetrahydrofuran (30 ml) and tert-butanol (90 ml) was added powdered potassium hydroxide (1.95 g, 34.8 mmol). The mixture was stirred at room temperature for 6 h, then 1M hydrochloric acid (40 ml) and ethyl acetate (100 ml) were added. The organic layer was washed with water and dried over sodium sulfate. Concentration in vacuo afforded the acid (Vd; Z═CO₂H) (13.1 g, 96%) as an almost colorless oil.

### Example 4

### 3-{(2S,3R)-1-(4-fluorophenyl)-2-[4-(4-methoxybenzyloxy)phenyl]-4-oxoazetidin-3-yl}propionic acid (Vg; Z═CO₂H)

To a solution of methyl 3-{(2S,3R)-1-(4-fluorophenyl)-2-[4-(4-methoxybenzyloxy)phenyl]-4-oxoazetidin-3-yl}propionate (Vg; Z═CO₂Me) (2.7 g, 4.31 mmol) in tetrahydrofuran (2 ml) and tert-butanol (6 ml) was added powdered potassium hydroxide (0.48 g, 8.63 mmol). The mixture was stirred at room temperature for 0.5 h, then 1M hydrochloric acid (10 ml) and ethyl acetate (20 ml) were added. The organic layer was washed with water and dried over sodium sulfate. Concentration in vacuo afforded the acid (Vg; Z═CO₂H) (1.78 g, 94%) as a yellow colored viscous oil.

### Example 5

### 3-{(3R,4S)-1-(4-fluorophenyl)-2-oxo-4-[4-(trityloxy)phenyl]azetidin-3-yl}propionic acid (Vh; Z═CO₂H)

To a solution of methyl 3-{(3R,4S)-1-(4-fluorophenyl)-2-oxo-4-[4-(trityloxy)phenyl]azetidin-3-yl}propionate (Vh; Z═CO₂Me) (6.0 g, 8.9 mmol) in tetrahydrofuran (10 ml) and tert-butanol (20 ml) was added powdered potassium hydroxide (0.6 g, 8.9 mmol). The mixture was stirred at room temperature for 19 h, then additional amount of powdered potassium hydroxide (0.15 g, 2.2 mmol) was added and stirring continued for 2 h. 1M hydrochloric acid (5 ml) and ethyl acetate (18 ml) were added, the organic layer was washed with water and dried over sodium sulfate. Concentration in vacuo afforded the acid (Vh; Z═CO₂H) (5.2 g, 88%) as an almost colorless foam.

### Example 6

### 3-{(2S,3R)-2-[4-(4-bromobenzyloxy)phenyl]-1-(4-fluorophenyl)-4-oxoazetidin-3-yl}propionyl chloride (Vc; Z═COCl)

To a solution of 3-{(2S,3R)-2-[4-(4-bromobenzyloxy)phenyl]-1-(4-fluorophenyl)-4-oxoazetidin-3-yl}propionic acid (Vc; Z═CO₂H) (9.2 g, 18.4 mmol) in dichloromethane (16 ml) was added 2M solution of oxalyl chloride (14 ml, 28.0 mmol) in dichloromethane and the mixture was stirred at room temperature for 18 h. Concentration in vacuo gave the acid chloride (Vc; Z═COCl) as a viscous amber colored oil.

### Example 7

### 3-{(2S,3R)-2-[4-(4-chlorobenzyloxy)phenyl]-1-(4-fluorophenyl)-4-oxoazetidin-3-yl}propionyl chloride (Vd; Z═COCl)

To a solution of 3-{(2S,3R)-2-[4-(4-chlorobenzyloxy)phenyl]-1-(4-fluorophenyl)-4-oxoazetidin-3-yl}propionic acid (Vd; Z═CO₂H) (13.0 g, 27.8 mmol) in dichloromethane (37 ml) was added 2M solution of oxalyl chloride (22.7 ml, 45.4 mmol) in dichloromethane and the mixture was stirred at room temperature for 18 h. Concentration in vacuo gave the acid chloride (Vd; Z═COCl) as a viscous brown colored oil.

### Example 8

### 3-{(2S,3R)-1-(4-fluorophenyl)-2-[4-(4-methoxybenzyloxy)phenyl]-4-oxoazetidin-3-yl}propionyl chloride (Vg; Z═COCl)

To a solution of 3-{(2S,3R)-1-(4-fluorophenyl)-2-[4-(4-methoxybenzyloxy)phenyl]-4-oxoazetidin-3-yl}propionic acid (Vg; Z═CO₂H) (2.6 g, 5.74 mmol) in dichloromethane (7 ml) was added 2M solution of oxalyl chloride (4.4 ml, 8.8 mmol) in dichloromethane and the mixture was stirred at room temperature for 18 h. Concentration in vacuo gave the acid chloride (Vg; Z═COCl) as a viscous amber colored oil.

### Example 9

### 3-{(3R,4S)-1-(4-fluorophenyl)-2-oxo-4-[4-(trityloxy)phenyl]azetidin-3-yl}propionyl chloride (Vh; Z═COCl)

To a solution of 3-{(3R,4S)-1-(4-fluorophenyl)-2-oxo-4-[4-(trityloxy)phenyl]azetidin-3-yl}propionic acid (Vh; Z═CO₂H) (5.1 g, 7.7 mmol) in dichloromethane (15 ml) was added 2M solution of oxalyl chloride (5.7 ml, 11.4 mmol) in dichloromethane and the mixture was stirred at room temperature for 18 h. Concentration in vacuo gave the acid chloride (Vh; Z═COCl) as a viscous yellow colored oil.

### Example 10

### (3R,4S)-4-[4-(4-bromobenzyloxy)phenyl]-1-(4-fluorophenyl)-3-[3-(4-fluorophenyl)-3-oxopropyl]azetidin-2-one (IIc)

To a solution of dried zinc chloride (2.68 g, 19.4 mmol) in tetrahydrofuran (20 ml) was added dropwise 1M solution of 4-fluorophenylmagnesium bromide in tetrahydrofuran (19.4 ml) at 4° C. while stirring. Tetrakis(triphenylphosphine)palladium (1.1 g, 0.96 mmol) was added to the resulting suspension of 4-fluorophenylzinc chloride at 10° C., followed by a solution of 3-{(2S,3R)-2-[4-(4-bromobenzyloxy)phenyl]-1-(4-fluorophenyl)-4-oxoazetidin-3-yl}propionyl chloride (Vc; Z═COCl) (9.2 g, 17.4 mmol) in tetrahydrofuran (17 ml) and the cooling bath was removed. After 7 h of stirring, 1M hydrochloric acid (17 ml) and ethyl acetate (250 ml) were added, the organic layer was washed with water and dried over sodium sulfate. Concentration afforded an oil (9.5 g) which was purified by silica gel chromatography with toluene/isopropanol (200/1). Ketone IIc was obtained as an amber colored oil.

### Example 11

### (3R,4S)-4-[4-(4-chlorobenzyloxy)phenyl]-1-(4-fluorophenyl-3-[3-(4-fluorophenyl)-3-oxopropyl]azetidin-2-one (IId)

To a solution of dried zinc chloride (3.88 g, 27 mmol) in tetrahydrofuran (25 ml) was added dropwise 1M solution of 4-fluorophenylmagnesium bromide in tetrahydrofuran (72 ml) at 0° C. while stirring. Tetrakis(triphenylphosphine)palladium (1.57 g, 1.36 mmol) was added to the resulting suspension of 4-fluorophenylzinc chloride (27 mmol) at 4° C., followed by a solution of 3-{(2S,3R)-2-[4-(4-chlorobenzyloxy)phenyl]-1-(4-fluorophenyl)-4-oxoazetidin-3-yl}propionyl chloride (Vd; Z═COCl) (12.0 g, 25 mmol) in tetrahydrofuran (20 ml) and the cooling bath was removed. After 3 h of stirring 1M hydrochloric acid (25 ml) and ethyl acetate (100 ml) were added, the organic layer was washed with water and dried over sodium sulfate. Concentration afforded a brown colored oil (12.7 g) which was purified by silica gel chromatography with toluene/isopropanol (200/1). Ketone IId was obtained as a brown colored oil.

### Example 12

### (3R,4S)-1-(4-fluorophenyl)-3-[3-(4-fluorophenyl)-3-oxopropyl]-4-[4-(4-methoxybenzyloxy)phenyl]azetidin-2-one (IIg)

To a solution of dried zinc chloride (0.47 g, 3.5 mmol) in tetrahydrofuran (3.5 ml) was added dropwise 1M solution of 4-fluorophenylmagnesium bromide in tetrahydrofuran (3.5 ml) at 4° C. while stirring. Tetrakis(triphenylphosphine)palladium (0.19 g, 0.16 mmol) was added to the resulting suspension of 4-fluorophenylzinc chloride at 0° C., followed by a solution of 3-{(2S,3R)-1-(4-fluorophenyl)-2-[4-(4-methoxybenzyloxy)phenyl]-4-oxoazetidin-3-yl}propionyl chloride (Vg; Z═COCl) (1.5 g, 2.89 mmol) in tetrahydrofuran (3.2 ml) and the cooling bath was removed. After 4 h of stirring 1M hydrochloric acid (2.3 ml) and ethyl acetate (50 ml) were added, the organic layer was washed with water, dried over sodium sulfate and concentrated to obtain the crude product IIg (1.1 g) as a brown colored oil.

### Example 13

### (3R,4S)-1-(4-fluorophenyl)-3-[3-(4-fluorophenyl)-3-oxopropyl]-4-[4-(trityloxy)phenyl]azetidin-2-one (IIh)

To a solution of dried zinc chloride (1.02 g, 7.3 mmol) in tetrahydrofuran (7 ml) was added dropwise 1M solution of 4-fluorophenylmagnesium bromide in tetrahydrofuran (7.3 ml) at 0° C. while stirring. Tetrakis(triphenylphosphine)palladium (0.41 g, 0.35 mmol) was added to the resulting suspension of 4-fluorophenylzinc chloride (7.3 mmol) at 4° C., followed by a solution of 3-{(3R,4S)-1-(4-fluorophenyl)-2-oxo-4-[4-(trityloxy)phenyl]-azetidin-3-yl}propionyl chloride (Vh; Z═COCl) (4.0 g, 7.0 mmol) in tetrahydrofuran (3.2 ml) and the cooling bath was removed. After 3 h of stirring 0.1M acetic acid (20 ml) and ethyl acetate (50 ml) were added, the organic layer was washed with water, dried over sodium sulfate and concentrated to obtain the product IIh (3.43 g) as a brown oil.

### Example 14

### 4-(4,6-dimethoxy-1,3,5-triazin-2-yl)-4-methylmorpholinium chloride (DMT-MM)

N-methylmorpholine (5.4 mL, 49.1 mmol) was added to a solution of 2-chloro-4,6-dimehoxy-1,3,5-triazine (9.53 g, 54.3 mmol) in THF (150 mL) at room temperature. A white solid appeared within several minutes. After stirring for 30 min at rt, the solid was collected by suction and washed with THF and dried to give DMT-MM (13.08, 96.3%) as a white solid.

### Example 15

### 3-((2S,3R)-2-(4-(benzyloxy)phenyl)-1-(4-fluorophenyl)-4-oxoazetidin-3-yl)-N-methoxy-N-methylpropanamide (Vb, Z═CON(Me)OMe)

### Procedure 1.

To a solution of 3-((2S,3R)-2-(4-(benzyloxy)phenyl)-1-(4-fluorophenyl)-4-oxoazetidin-3-yl)propanoic acid (17.7 g, 42.2 mmol), N,O-dimethylhydroxylamine hydrochloride (6.19 g, 63.4 mmol), and N-methylmorpholine (9.6 mL, 87.3 mmol) in methanol (350 mL), was added DMT-MM (14.1 g, 51.0 mmol) at room temperature. The reaction mixture was stirred until disappearance of the acid, as determined using TLC. After removal of the solvent under reduced pressure, the residue was extracted with ethyl acetate (200 mL). The organic layer was washed successively with saturated NaHCO₃ solution (200 mL), 1 M HCl (200 mL), water (200 mL), and brine (100 mL), then dried over Na₂SO₄ and concentrated to afford 3-((2S,3R)-2-(4-(benzyloxy)phenyl)-1-(4-fluorophenyl)-4-oxoazetidin-3-yl)-N-methoxy-N-methylpropanamide (19.68 g, 100%) as a viscous yellow coloured oil.

¹H NMR (CDCl₃) δ/ppm: 2.24 (m, 2H), 2.65 (m, 2H), 3.12 (m, 4H), 3.62 (s, 3H), 4.66 (d, 1H), 5.04 (s, 2H), 6.88-6.97 (m, 4H), 7.21-7.26 (m, 4H), 7.33-7.40 (m, 5H).

HRMS (Q-TOF), m/z: 463.2020 (MH⁺, calcd for C₂₇H₂₈N₂O₄F 463.2033)

### Procedure 2.

To a suspension of N,O-dimethylhydroxylamine hydrochloride (6.19 g, 63.4 mmol) in 150 mL dichloromethane at 0° C. was added dimethylaluminium chloride (1 M in hexane, 63.4 mL, 63.4 mmol). After stirring at room temperature for 1 hr, a solution of methyl 3-{(2S,3R)-2-[4-benzyloxyphenyl]-1-(4-fluorophenyl)-4-oxoazetidin-3-yl}propionate (13.74 g, 31.7 mmol) in 50 mL dichloromethane was added and resulting mixture was stirred overnight. Upon completion, saturated aq. NH₄Cl (200 mL) was added. The organic layer was separated, dried using MgSO₄, filtered and evaporated to afford 3-((2S,3R)-2-(4-(benzyloxy)phenyl)-1-(4-fluorophenyl)-4-oxoazetidin-3-yl)-N-methoxy-N-methylpropanamide (13.68 g, 93.3%) as a viscous yellow coloured oil.

### Procedure 3.

To a solution of the 3-{(2S,3R)-2-[4-(4-bromobenzyloxy)phenyl]-1-(4-fluorophenyl)-4-oxoazetidin-3-yl}propionyl chloride (48.16 g, 0.11 mol) in dichloromethane (250 mL) was added N-methoxy-N-methylamine hydrochloride (11 g, 0.11 mol) and triethyl amine (30 mL, 0.22 mol) at 0° C. After stirring at room temperature for 4 h, the reaction mixture was diluted with ether (500 mL) and successively washed with water, dilute aqueous sodium hydrogen sulfate and brine, dried over anhydrous magnesium sulfate, filtered and concentrated to dryness to afford 3-((2S,3R)-2-(4-(benzyloxy)phenyl)-1-(4-fluorophenyl)-4-oxoazetidin-3-yl)-N-methoxy-N-methylpropanamide (46.25 g, 91%), which was used without further purification.

Procedure 4. To a suspension of N,O-dimethylhydroxylamine hydrochloride (6.74 g, 70.5 mmol) in 100 mL toluene at 0° C. was added diethylaluminium chloride (1.8 M in toluene, 39 mL, 70.2 mmol). After stirring at 0° C. for 30 min, a solution of methyl 3-{(2S,3R)-2-[4-benzyloxyphenyl]-1-(4-fluorophenyl)-4-oxoazetidin-3-yl}propionate (10.10 g, 23.3 mmol) in 100 mL toluene was added and resulting mixture was stirred at 0° C. for 1 hr. Upon completion, saturated aq. NH₄Cl (100 mL) was added. The organic layer was separated, dried using Na₂SO₄, filtered and evaporated to afford 3-((2S,3R)-2-(4-(benzyloxy)phenyl)-1-(4-fluorophenyl)-4-oxoazetidin-3-yl)-N-methoxy-N-methylpropanamide (10.4 g, 96.5%) as a viscous yellow coloured oil.
Procedure 5. To a suspension of methyl 3-{(2S,3R)-2-[4-benzyloxyphenyl]-1-(4-fluorophenyl)-4-oxoazetidin-3-yl}propionate (1 g, 2.31 mmol) and N,O-dimethylhydroxylamine hydrochloride (0.34 g, 3.46 mmol) in THF (20 mL) at −10° C. was added a 2.0 M solution of isopropylmagnesium chloride in THF (3.5 mL, 7.0 mmol) over a 30 min period. The reaction was stirred at −10° C. for 1 hr. Upon completion by TLC, saturated aq. NH₄Cl (20 mL) and ethyl acetate (20 mL) were added. The organic layer was separated, and the aqueous layer was extracted with ethyl acetate (2×20 mL). The organic layers were combined, washed with saturated aq. NaHCO₃ (20 mL), water (20 mL) and dried over Na₂SO₄, filtered and evaporated to afford 3-((2S,3R)-2-(4-(benzyloxy)phenyl)-1-(4-fluorophenyl)-4-oxoazetidin-3-yl)-N-methoxy-N-methylpropanamide (0.98 g, 91.7%) as a viscous yellow coloured oil.

### Example 16

### (3R,4S)-3-(3-(1H-benzo[d][1,2,3]triazol-1-yl)-3-oxopropyl)-4-(4-(benzyloxy)phenyl)-1-(4-fluorophenyl)azetidin-2-one (Vb, Z═CO-benzotriazol-1-yl)

To a solution of the 3-{(2S,3R)-2-[4-(4-bromobenzyloxy)phenyl]-1-(4-fluorophenyl)-4-oxoazetidin-3-yl}propionyl chloride (9.50 g, 22.6 mmol) in dichloromethane (30 mL) was added benzotriazole (7.62 g, 64.0 mmol) at rt. After stirring at room temperature overnight, the reaction mixture was diluted with dichloromethane (50 mL) and successively washed with water, dilute aqueous sodium hydrogen sulfate and brine, dried over anhydrous magnesium sulfate, filtered and concentrated to dryness to afford (3R,4S)-3-(3-(1H-benzo[d][1,2,3]triazol-1-yl)-3-oxopropyl)-4-(4-(benzyloxy)phenyl)-1-(4-fluorophenyl)azetidin-2-one (10.01 g, 85.1%), which was used without further purification.

¹H NMR (CDCl₃) δ/ppm: 2.52 (m, 2H), 3.30 (m, 2H), 3.69 (m, 1H), 4.77 (d, 1H), 5.00 (s, 2H), 6.88-6.95 (m, 4H), 7.22-7.64 (m, 10H), 77.91 (m, 1H), 8.08-8.26 (M, 2H).

### Example 17

### (3R,4S)-4-(4-(benzyloxy)phenyl)-1-(4-fluorophenyl)-3-(3-(4-fluorophenyl)-3-oxopropyl)azetidin-2-one (IIb)

A round-bottomed flask was charged with a mixture of Na₂CO₃ (0.612 g, 5.8 mmol), Pd (OAc)₂ (15 mg, 0.067 mmol), [bmim][PF₆] (10 g), and H₂O (10 g). The solution was heated to 60° C. with stirring, and 3-{(2S,3R)-2-[4-(4-bromobenzyloxy)phenyl]-1-(4-fluorophenyl)-4-oxoazetidin-3-yl}propionyl chloride (1.575 g, 3.6 mmol) and 4-fluorophenylboronic acid (0.606 g, 4.3 mmol) were added. The mixture was stirred at 60° C. for the 24 hrs and cooled to room temperature. The suspension was extracted with tetrbutyl methyl ether (20 mL) four times. The combined organic phase was concentrated, and further purification of the product was achieved by flash chromatography on a silica gel column to give a title compound (1.49 g, 83.3%).

### Example 18

### (3R,4S)-4-(4-(benzyloxy)phenyl)-1-(4-fluorophenyl)-3-(3-(4-fluorophenyl)-3-oxopropyl)azetidin-2-one (IIb)

To a solution of bis[2-(N,N,-dimethylaminoethyl)]ether (0.90 mL, 4.7 mmol) in THF (10 mL) was added 4-fluorophenylmagnesium bromide (4.7 mL, 4.7 mmol, 1 M solution in THF) at 0° C. The mixture was stirred at 0-5° C. for 15 min. This mixture was slowly added to a solution of 3-{(2S,3R)-2-[4-(4-bromobenzyloxy)phenyl]-1-(4-fluorophenyl)-4-oxoazetidin-3-yl}propionyl chloride (1.575 g, 3.6 mmol) in THF (10 mL) at −10° C. over 15 min, and the resulting mixture was stirred at −10° C. for 30 min. The mixture was then quenched with aqueous ammonium chloride. After extraction of the mixture with EtOAc, the extract was dried over MgSO₄ and concentrated. The residue was purified by chromatography on silica gel to give title compound (1.32 g, 73.7%).

### Example 19

### (3R,4S)-4-(4-(benzyloxy)phenyl)-1-(4-fluorophenyl)-3-(3-(4-fluorophenyl)-3-oxopropyl)azetidin-2-one (IIb)

To a solution of 3-((2S,3R)-2-(4-(benzyloxy)phenyl)-1-(4-fluorophenyl)-4-oxoazetidin-3-yl)-N-methoxy-N-methylpropanamide (5.53 g, 11.9 mmol) in dry THF (20 mL) cooled to 0° C., was added dropwise 1 M solution of 4-fluorophenylmagnesium bromide in THF (18 mL, 18 mmol). The resulting suspension was stirred at 0° C. for 3 hrs. Thereafter, 1 M hydrochloric acid (50 mL) and ethyl acetate (20 mL) were added, layers separated, and the aqueous layer extracted with ethyl acetate (2×20 mL). The combined organic layers were washed with saturated solution of NaHCO₃ (50 mL) and brine (50 mL), then dried over Na₂SO₄, and concentrated to afford crude (3R,4S)-4-(4-(benzyloxy)phenyl)-1-(4-fluorophenyl)-3-(3-(4-fluorophenyl)-3-oxopropyl)azetidin-2-one as a yellow coloured oil (5.43 g, 91.7%)

### Example 20

### (3R,4S)-4-(4-(benzyloxy)phenyl)-1-(4-fluorophenyl)-3-(3-(4-fluorophenyl)-3-oxopropyl)azetidin-2-one (IIb)

To a solution of (3R,4S)-3-(3-(1H-benzo[d][1,2,3]triazol-1-yl)-3-oxopropyl)-4-(4-(benzyloxy)phenyl)-1-(4-fluorophenyl)azetidin-2-one (1.0 g, 2.3 mmol) in dry THF (10 mL) cooled to −10° C., was added dropwise 1 M solution of 4-fluorophenylmagnesium bromide in THF (6 mL, 6 mmol). The resulting suspension was stirred at −10° C. for 3 hrs. Thereafter, 1 M hydrochloric acid (50 mL) and ethyl acetate (20 mL) were added, layers separated, and the aqueous layer extracted with ethyl acetate (2×20 mL). The combined organic layers were washed with saturated solution of NaHCO₃ (50 mL) and brine (50 mL), then dried over Na₂SO₄, and concentrated to afford crude (3R,4S)-4-(4-(benzyloxy)phenyl)-1-(4-fluorophenyl)-3-(3-(4-fluorophenyl)-3-oxopropyl)azetiin-2-one as a yellow coloured oil (0.97 g, 84.7%).

### Example 21

### (3R,4S)-4-(4-(benzyloxy)phenyl)-1-(4-fluorophenyl)-3-(3-(4-fluorophenyl)-3-oxopropyl)azetidin-2-one (IIb)

To a suspension of methyl 3-{(2S,3R)-2-[4-benzyloxyphenyl]-1-(4-fluorophenyl)-4-oxoazetidin-3-yl}propionate (10.0 g, 23.1 mmol) and N,O-dimethylhydroxylamine hydrochloride (3.38 g, 34.65 mmol) in THF (200 mL) at −10° C. was added a 2.0 M solution of isopropylmagnesium chloride in THF (35.0 mL, 70.0 mmol) over a 1 hr period. The reaction was stirred at −10° C. for 1 hr. Upon completion by TLC, 1.0 M solution of 4-fluorophenylmagnesium bromide in THF (40 mL, 40.0 mmol) was added over a 1 hr period. The resulting suspension was stirred at −10° C. for 2 hrs. Thereafter, saturated aq. Solution of NH₄Cl (200 mL) and ethyl acetate (100 mL) were added, layers separated, and the aqueous layer extracted with ethyl acetate (2×100 mL). The combined organic layers were washed with saturated solution of NaHCO₃ (200 mL) and brine (200 mL), then dried over Na₂SO₄, and concentrated to afford crude (3R,4S)-4-(4-(benzyloxy)phenyl)-1-(4-fluorophenyl)-3-(3-(4-fluorophenyl)-3-oxopropyl)azetidin-2-one as a yellow coloured oil (10.79 g, 94.0%).

### Example 22

### Transfer hydrogenation of ketone (IIb)

The Ru-complex was prepared from [RuCl₂(mesitylene)]₂ (11.5 mg, 40 μmmol Ru at.) and (1S,2S)—N-piperidylsulfamoyl-1,2-diphenylethylenediamine (17 mg, 48 μmol) by heating in acetonitrile (2 ml) at 80° C. for 30 min. The Ru-complex solution and HCO₂H-Et₃N (5:2, 2 ml) were then added over 24 h in 5 portions to (IIb) (2.50 g, 5.0 mmol) in acetonitrile (5 ml) stirred at 40° C. The mixture was partitioned between ethyl acetate (20 ml) and water (20 ml), the organic layer washed with brine (20 ml), dried over Na₂SO₄, and filtered through a bed of silica gel. The residue of concentration was recrystallized from iso-propyl ether, then from ethanol to afford 2.27 g (90.5%) of the alcohol (Ib) with dr=94:6 (determined by ¹⁹F NMR (CDCl₃) using 1.5 mol equiv. Eu(hfc)₃).

### Example 23

### Transfer hydrogenation of ketone (IIa)

The Ru-complex was prepared from [RuCl₂(mesitylene)]₂ (2.1 mg, 7.2 μmol Ru at.) and (1S,2S)—N-piperidylsulfamoyl-1,2-diphenylethylenediamine (3.2 mg, 8.9 μmol) by heating in (CH₂Cl)₂ (0.5 ml) at 80° C. for 30 min. The Ru-complex solution and HCO₂H-Et₃N (5:2, 210 μl) were then added in portions over 24 h to (IIa) (150 mg, 0.37 mmol) in (CH₂Cl)₂ (0.5 ml) stirred at 40° C. The mixture was partitioned between ethyl acetate (5 ml) and water (5 ml), the organic layer washed with brine (5 ml), dried over Na₂SO₄, and filtered through a bed of silica gel. The residue of concentration (134 mg) was recrystallized from ethanol-water (4:1) to afford the product (Ia) with dr>99:1 (determined by ¹⁹F NMR (CDCl₃) using 1.5 mol equiv. Eu(hfc)₃).

### Example 24

### O-Deprotection of alcohol (Ib)

A mixture of alcohol (Ib) (2.19 g, 4.38 mmol, dr=94:6) and 10% Pd—C (160 mg) in ethanol/ethyl acetate (2:1, 35 ml) was hydrogenated at 30 psi of H₂ for 10 h and then filtered through Celite. The residue of concentration was recrystallized from ethanol-water (4:1) to afford 1.27 g (71%) of product (Ia) melting from 160 to 163° C. with dr>99:1 (determined by ¹⁹F NMR (CDCl₃) using 1.5 mol equiv. Eu(hfc)₃).

### Example 25

### Methyl 3-[(2S,3R)-1-(4-fluorophenyl)-2-(4-hydroxyphenyl)-4-oxoazetidin-3-yl]propionate (Va; Z═CO₂Me)

To a solution of methyl 3-{(2S,3R)-2-[4-(benzyloxy)phenyl]-1-(4-fluorophenyl)-4-oxoazetidin-3-yl}propionate (Vb; Z═CO₂Me) (50 g, 115 mmol) in ethyl acetate (90 mL) was added 10% palladium on carbon (4 g, with 49.6% of water, Engelhard). The reaction mixture was shaken in a pressure bottle under the pressure of hydrogen gas (3.5 bar) for 20 h. Catalyst was removed by filtration through a filter aid and washed with ethyl acetate (10 ml). The resulting solution was dried over sodium sulfate and concentrated. The solid residue was purified by recrystallization in methanol/water (5/1) to yield the ester (Va; Z═CO₂Me) (38.4 g, 97%) as a white solid with m. p. 136-137° C.

### Example 26

### Methyl 3-{(2S,3R)-2-[4-(4-bromobenzyloxy)phenyl]-1-(4-fluorophenyl)-4-oxoazetidin-3-yl}propionate (Vc; Z═CO₂Me)

A mixture of methyl 3-[(2S,3R)-1-(4-fluorophenyl)-2-(4-hydroxyphenyl)-4-oxoazetidin-3-yl]propionate (Va; Z═CO₂Me) (10.0 g, 29 mmol), 4-bromobenzyl bromide (11.0 g, 41 mmol), anhydrous potassium carbonate (40.0 g, 0.29 mol) and tetrabutylammonium iodide (1.00 g, 3 mmol) in acetone (30 ml) was stirred under reflux for 3.5 h. The mixture was cooled to room temperature, filtered and concentrated in vacuo. The residue was purified by silica gel chromatography with toluene/ethyl acetate (5/1) to give pure ester (Vc; Z═CO₂Me) (11.5 g, 85%) as a white colored solid.

### Example 27

### Methyl 3-{(2S,3R)-2-[4-(4-chlorobenzyloxy)phenyl]-1-(4-fluorophenyl)-4-oxoazetidin-3-yl}propionate (Vd; Z═CO₂Me)

A mixture of methyl 3-[(2S,3R)-1-(4-fluorophenyl)-2-(4-hydroxyphenyl)-4-oxoazetidin-3-yl]propionate (Va; Z═CO₂Me) (1 g, 2.9 mmol), 4-chlorobenzyl chloride (0.7 g, 3.2 mmol), anhydrous potassium carbonate (2 g, 15 mmol) and tetrabutylammonium iodide (0.01 g, 0.03 mmol) in acetone (4 ml) was stirred under reflux for 5 h. The mixture was cooled to room temperature, filtered and concentrated in vacuo. The residue was purified by silica gel chromatography with toluene/ethyl acetate (3/1) to give pure ester (Vd; Z═CO₂Me) (0.8 g, 60%) as an amber colored oil.

### Example 28

### Methyl 3-{(2S,3R)-1-(4-fluorophenyl)-2-[4-(4-nitrobenzyloxy)phenyl]-4-oxoazetidin-3-yl}propionate (Ve; Z═CO₂Me)

A mixture of methyl 3-[(2S,3R)-1-(4-fluorophenyl)-2-(4-hydroxyphenyl)-4-oxoazetidin-3-yl]propionate (Va; Z═CO₂Me) (100 mg, 0.29 mmol), 4-nitrobenzyl chloride (63 mg, 0.364 mmol), anhydrous potassium carbonate (102 mg, 0.56 mmol) and tetrabutylammonium iodide (18 mg, 0.05 mmol) in acetone (4 ml) was stirred under reflux for 7 h. The mixture was cooled to room temperature, filtered and concentrated in vacuo. The residue was purified by silica gel chromatography with toluene/ethyl acetate (9/1) to give pure ester (Ve; Z═CO₂Me) (69 mg, 50%) as an amber colored oil.

### Example 29

### Methyl 3-{(2S,3R)-2-[4-(biphenyl-4-ylmethoxy)phenyl]-1-(4-fluorophenyl)-4-oxoazetidin-3-yl}propionate (Vf; Z═CO₂Me)

A mixture of methyl 3-[(2S,3R)-1-(4-fluorophenyl)-2-(4-hydroxyphenyl)-4-oxoazetidin-3-yl]propionate (Va; Z═CO₂Me) (1.0 g, 2.9 mmol), 4-phenylbenzyl chloride (0.61 g, 3.0 mmol), anhydrous potassium carbonate (1.0 g, 7.2 mmol) and tetrabutylammonium iodide (0.01 g, 0.03 mmol) in acetone (10 ml) was stirred under reflux for 5 h. The mixture was cooled to room temperature, filtered and concentrated in vacuo. The residue was purified by silica gel chromatography with toluene/ethyl acetate (9/1) to give pure ester (Vf; Z═CO₂Me) (0.80 g, 60%) as a brown colored oil.

### Example 30

### Methyl 3-{(2S,3R)-1-(4-fluorophenyl)-2-[4-(4-methoxybenzyloxy)-phenyl]-4-oxoazetidin-3-yl}propionate (Vg; Z═CO₂Me)

A mixture of methyl 3-[(2S,3R)-1-(4-fluorophenyl)-2-(4-hydroxyphenyl)-4-oxoazetidin-3-yl]propionate (Va; Z═CO₂Me) (100 mg, 0.29 mmol), 4-methoxybenzyl bromide (75 mg, 0.364 mmol), anhydrous potassium carbonate (200 mg, 1.45 mmol) and tetrabutylammonium iodide (10 mg, 0.03 mmol) in acetone (1 ml) was stirred under reflux for 4 h. The mixture was cooled to room temperature, filtered and concentrated in vacuo. The residue was purified by silica gel chromatography with toluene/ethyl acetate (9/1) to give pure ester (Vg; Z═CO₂Me) (105 mg, 78%).

### Example 31

### Methyl 3-{(3R,4S)-1-(4-fluorophenyl)-2-oxo-4-[4-(trityloxy)phenyl]-azetidin-3-yl}propionate (Vh; Z═CO₂Me)

A mixture of methyl 3-[(2S,3R)-1-(4-fluorophenyl)-2-(4-hydroxyphenyl)-4-oxoazetidin-3-yl]propionate (Va; Z═CO₂Me) (5 g, 20.5 mmol), triethylamine (3 ml), triphenylchloromethane (6.6 g, 23.7 mmol) in acetone (19 ml) was stirred at room temperature for 3 h. Water (2.6 ml) was added to a suspension, cooled to 15° C. and filtered. The precipitate was washed with 50% aq. acetone (1.9 ml) and water (4.3 ml). The solid was suspended in 50% aq. acetone (18 ml) and stirred at 15-18° C. for 1 h. The precipitate (10.63 g) was filtered, washed with water and dried in a vacuum oven at 45° C. over P₂O₅ for 10 h. Crude product was purified by silica gel chromatography with hexane/ethyl acetate (10/1) to give pure ester (Vh; Z═CO₂Me) (6.64 g, 55%) as a white solid with mp 138-140° C.

### Example 32

### Methyl 3-{(2S,3R)-2-[4-(tert-butyldimethylsilyloxy)phenyl]-1-(4-fluorophenyl)-4-oxoazetidin-3-yl}propionate (Vi; Z═CO₂Me)

A mixture of methyl 3-[(2S,3R)-1-(4-fluorophenyl)-2-(4-hydroxyphenyl)-4-oxoazetidinyl]propionate (Va; Z═CO₂Me) (500 mg, 1.46 mmol), tert-butyldimethylsilyl chloride (850 mg, 3.64 mmol) and imidazole (11 mg, 0.03 mmol) in N,N-dimethylformamide (10 ml) was stirred at 35° C. for cca. 5 h. The mixture was cooled to room temperature, then 5% solution of sodium hydrogencarbonate (10 ml) and diethyl ether were added. The organic layer was washed with water, dried over sodium sulfate and concentrated in vacuo. The residue was purified by silica gel chromatography with toluene/ethyl acetate (9/1) to give pure ester (Vi; Z═CO₂Me) (541 mg, 71%) as a brown colored oil.

### Example 33

### Methyl 3-{(3R,4S)-1-(4-fluorophenyl)-2-oxo-4-[4-(tetrahydro-2H-pyran-2-yloxy)phenyl]azetidin-3-yl}propanoate (Vk; Z═CO₂Me)

A mixture of methyl 3-[(2S,3R)-1-(4-fluorophenyl)-2-(4-hydroxyphenyl)-4-oxoazetidinyl]propionate (Va; Z═CO₂Me) (1.0 g, 2.91 mmol), pyridinium toluene-4-sulfonate (0.92 g, 2.94 mmol) and 3,4-dihydro-2H-pyran (0.43 g, 5.6 mmol) in methylene chloride (40 ml) was stirred at room temperature for 17 h. Then 5% solution of sodium hydrogencarbonate (10 ml) and diethyl ether (40 ml) were added, the organic phase was washed with water, dried over sodium sulfate and concentrated in vacuo. Ester (Vk; Z═CO₂Me) was obtained (0.81 g) as an almost colorless oil.

### Example 34

### Crystallization of ezetimibe (anhydro form A)

1 g of ezetimibe (anhydro form A) was dissolved in a selected solvent by heating at reflux. The choice and volume of the solvent is shown in Table 1. The resulting solution was allowed to cool to room temperature with magnetic stirring or further down to 0° C. The ultimate temperature of cooling (Tᵤ) is also indicated in Table 1. The solid was collected by filtration, suction dried for cca. 10 min, then dried in a desiccator at 20° C. (RH below 15%) for 16 h and analyzed. Precipitates obtained from propionitrile and α,α,α-trifluorotoluene were dried in a vacuum oven at 50° C. for 16 h. XRPD results are given in Table 1. All samples melted within the range of 156-164° C., except for the sample obtained from tert-butanol (form S), which melted partially at 83-86° C., then resolidified and melted again at 156-160° C.

TABLE 1

| | Vol. | Tᵤ | Yield | |
|---|---|---|---|---|
| Solvent | (ml) | (° C.) | (%) | Resulting cryst. form |
| Isopropyl acetate | 3 | 20 | 61.5 | A |
| Butyl acetate | 3 | 0 | 56 | A |
| Nitromethane | 3 | 20 | 90 | A |
| Acetonitrile | 2 | 20 | 91 | A |
| Propionitrile | 3 | 0 | 34 | A |
| Toluene | 17 | 20 | 89 | A |
| Chlorobenzene | 6 | 20 | 76.5 | A |
| α,α,α-Trifluorotoluene | 167 | 0 | 57 | A |
| Anisol | 1.9 | 20 | 92.5 | A |
| Cyclopentyl methyl ether | 1.9 | 20 | 90 | A |
| 2-Methyltetrahydrofuran | 1 | 20 | 85 | A |
| tert-Butyl methyl ether + | 22 | 20 | 92 | A |
| n-heptane | 25 | | | |
| tert-Butanol | 1.9 | 20 | 86 | S |
| Tetrachloroethylene | 75 | 20 | 90 | A |
| Acetic acid/water (6/1 v/v) | 3.5 | 20 | 78 | A/H ≈ 1/9 |
| Methanol + | 2.5 | 0 | 80.5 | H |
| water | 0.5 | | | |

### Example 35

### Crystallization of ezetimibe (hydrated form H)

1 g of ezetimibe (hydrated form H) was dissolved in a selected solvent by heating at reflux. The choice and volume of the solvent is shown in Table 2. The resulting solution was allowed to cool to room temperature with magnetic stirring. The solid was collected by filtration, suction dried for cca. 10 min, then air-dried at 20° C. (RH of 35-45%) till constant weight and analyzed. XRPD results are given in Table 2. All samples melted within the range of 156-164° C., except for the sample obtained from tert-butanol (form S), which melted first at 87-94° C., recrystallized at 96° C. and melted second at 159-162° C.

TABLE 2

| | Vol. | Yield | Resulting |
|---|---|---|---|
| Solvent | (ml) | (%) | cryst. form |
| Isopropyl acetate | 2.3 | 87 | H + A |
| Butyl acetate | 1.5 | 88.5 | H > A |
| Nitromethane | 1.4 | 94 | H >> A |
| Acetonitrile | 1.5 | 82.5 | H > A |
| Propionitrile | 1.4 | 60 | H >> A |
| Toluene | 16 | 92 | H + A |
| Chlorobenzene | 5.7 | 92.5 | H + A |
| Anisol | 2 | 76 | H + A |
| Cyclopentyl methyl ether | 2.4 | 81.5 | H + A |
| 2-Methyltetrahydrofuran | 1 | 53.5 | H + A |
| tert-Butyl methyl ether + | 22 | 99 | A |
| n-heptane | 25.5 | | |
| tert-Butanol | 1.9 | 89.5 | S |
| Acetic acid | 1.4 | 95.5 | H + A |

Explanation of symbols: H >> A: form A is present in traces; H > A: form A present in small amount; H + A: both forms present in substantial amounts by XRPD

### Example 36

### Crystallization of ezetimibe (anhydro form A) from ethanol

1 g of ezetimibe (anhydro form A) was dissolved in ethanol by heating at reflux. The grade and volume of the solvent is shown in Table 3. The resulting solution was magnetically stirred at room temperature for 1 h and at 0° C. for 2 h. The solid was collected by filtration, suction dried for cca. 10 min, then air-dried at 21° C. and 36% RH for 16 h, and analyzed by XRPD. Results are given in Table 3. Both samples melted within the range of 157-164° C.

TABLE 3

| | Vol. | Yield | Resulting |
|---|---|---|---|
| Solvent | (ml) | (%) | cryst. form |
| 96% Ethanol | 2 | 43 | A < H |
| Abs. ethanol | 2 | 42 | A > H |

Explanation of symbols: A < H: form A present in small amount; A > H: form H present in small amount

### Example 37

### Crystallization of ezetimibe by slow concentration of ethanol solution

0.50 g of ezetimibe (anhydro form A) was dissolved in 1 ml of warm ethanol. The clear solution was concentrated on a rotary evaporator at cca. 250 mbar starting pressure, which was gradually decreased to the ultimate pressure of cca. 50 mbar. The grade of ethanol and heating bath temperature are given in Table 4. Viscous oily residue formed initially, which solidified soon. When constant weight was achieved, the samples were analyzed directly by XRPD. Results are given in Table 4. All samples melted within the range of 158-162.5° C.

TABLE 4

| Ethanol | Bath T (° C.) | Resulting cryst. form |
|---|---|---|
| Abs. | 44 | A |
| Techn. grade | 44 | A + H |
| Abs. | 23 | A |
| Techn. grade | 23 | A + H |
| 96% | 23 | A + H |

Explanation of symbols: A + H: both forms present in substantial amounts by XRPD

### Example 38

### Crystallization of ezetimibe (anhydro form A) from aqueous methanol

27.0 g of ezetimibe (anhydro form A) was dissolved in a mixture of methanol (120 ml) and water (24 ml) by heating at reflux temperature. The resulting solution was allowed to cool to room temperature, then cooled in an ice bath for 30 min. The solid was collected by filtration, washed with an ice-cold methanol/water (2/1) mixture (54 ml) and air-dried at 20° C. and cca. 40% RH for 16 h. Hydrated form of ezetimibe H (25.78 g, 91.5%) with mp. 158-161° C. was obtained, which contained 4.5% water according to KF analysis. LOD experiments are given in Example 41.

### Example 39

### Crystallization of ezetimibe (hydrated form H) from aqueous tert-butanol

1.04 g of ezetimibe (hydrated form H) was dissolved in tert-butanol/water mixture (10/1, 5 ml) by heating at reflux temperature. The resulting solution was cooled to room temperature and stirred mechanically until thickening took place (cca. 1 h). The solid was collected by filtration and air-dried overnight. Hydrated form of ezetimibe H (0.77 g, 74.5%) with mp. 160-162° C. was obtained according to XRPD analysis, which contained 6.2% water by KF analysis and showed LOD of −5.5% at 130° C.

### Preparation of ezetimibe tert-butanol solvate (S)

### Example 40

### Crystallization of ezetimibe (anhydro form A) from tert-butanol

### Procedure 1.

5.06 g of ezetimibe (anhydro form A) was dissolved in tert-butanol (9.5 ml) by heating at reflux temperature. While stirred magnetically, the resulting solution was allowed to cool to room temperature. The solid was collected by filtration and dried in desiccator for 16 h. Pure S form of ezetimibe (tert-butanol solvate) (5.43 g) was obtained according to XRPD analysis, which melted first at 86-90° C., resolidified above 96° C. and melted second at 155-160° C. Sample was 99.2% pure by HPLC, contained 1.5% of water according to KF analysis, and showed LOD of −11.5% at 130° C. ¹H-NMR (DMSO-d₆): δ=1.11 (s, 6.0H, t-Bu), 1.6-1.9 (m, 4H, H-1′, H-2′), 3.08 (m, 1H, H-3), 4.20 (s, 0.7H, t-Bu-OH), 4.49 (m, 1H, H-3′), 4.80 (d, J=2.3 Hz, 1H, H-4), 5.29 (br d, J=2.7 Hz, 1H, OH-3′), 6.73-6.78 (m, 2H, Ar—H), 7.08-7.34 (m, 10H, Ar—H), 9.54 (br s, 1H, Ar—OH). Both NMR and KF analyses showed the structure of solvate to be ezetimibe.0.67 tert-BuOH.0.33 H₂O in this particular case.

### Procedure 2.

5.13 g of ezetimibe (anhydro form A) was dissolved in tert-butanol (12 ml) by heating at reflux temperature. While stirred mechanically, the resulting solution was allowed to cool to room temperature. The solid was collected by filtration and dried in desiccator for 16 h. Pure S form of ezetimibe (tert-butanol solvate) (5.64 g) was obtained according to XRPD analysis, which melted first at 86-90° C., resolidified above this temperature and melted second at 158-161° C. Sample was 99.4% pure by HPLC, contained 0.67% of water according to KF analysis and showed LOD of −12.0% at 130° C. within 4.5 min.

### Procedure 3.

Anhydrous ezetimibe (30 g) was dissolved in terc-butanol (204 mL) by heating suspension to 60° C., until clear sulution was obtained. The resulting sollution was allowed to cool to 33° C., when seeding crystals of ezetimibe form S were added. Crystallisation started, suspension was alloyed to cool to 28° C. and product was left to crystallize at this temperature for 18 hours. Dense suspension was recovered by filtration and pruduct was dried in vacuum dryer at 40° C. Yield: 34 g of ezetimibe form S.

### Example 41

### Crystallization of ezetimibe (hydrated form H) from tert-butanol

### Procedure 1.

5.01 g of ezetimibe (hydrated form H) was dissolved in tert-butanol (9 ml) by heating at reflux temperature. While stirred magnetically, the resulting solution was allowed to cool to room temperature. The solid was collected by filtration and air-dried for 3 d. Pure S form of ezetimibe (tert-butanol solvate) (5.34 g) was obtained according to XRPD analysis, which melted first at 87-90° C., resolidified above this temperature and melted second at 161-163° C. Sample was 99.8% pure by HPLC, contained 1.1% of water according to KF analysis, and showed LOD of −10.3% at 130° C.

### Procedure 2.

5.04 g of ezetimibe (hydrated form H) was dissolved in tert-butanol (9 ml) by heating at reflux temperature. While stirred mechanically, the resulting solution was allowed to cool to room temperature. No precipitation took place in this case even after 3 days, but after seeding with crystals of ezetimibe tert-butanol solvate crystallization did occur. The solid was collected by filtration and air-dried overnight. Pure S form of ezetimibe (tert-butanol solvate) (5.43 g) was obtained according to XRPD analysis, which melted first at 84-89° C., resolidified above this temperature and melted second at 162-163.5° C. Sample was 99.6% pure by HPLC, contained 0.95% of water according to KF analysis, and showed LOD of −13.1% at 130° C.

### Example 42

### Slurrying of ezetimibe (anhydro form A) in tert-butanol

### Procedure 1.

1.0 g of ezetimibe (anhydro form A) was slurried in tert-butanol (2.5 ml) at room temperature. While stirred magnetically the mixture thickened considerably within 2 h. The solid was collected by filtration and dried in desiccator for 3 d. Ezetimibe tert-butanol solvate (1.05 g) was obtained in admixture with a trace of anhydro form (S>>A) according to XRPD analysis, which melted first at 81-83° C., resolidified above this temperature and melted second at 152-155.5° C. The sample contained 0.48% of water according to KF analysis and showed LOD of −10.5% at 130° C.

### Procedure 2.

1.01 g of ezetimibe (anhydro form A) was slurried in tert-butanol (2.5 ml) at room temperature. While stirred mechanically the mixture thickened considerably within 7 h. The solid was collected by filtration and dried in desiccator for 3 d. Ezetimibe tert-butanol solvate (1.05 g) was obtained in admixture with a trace of anhydro form (S>>A) according to XRPD analysis, which melted partially at 87-90° C., resolidified above this temperature and melted second at 156-160° C. The sample contained 0.48% of water according to KF analysis and showed LOD of −11.8% at 130° C.

### Example 43

### Slurrying of ezetimibe (hydrated form H) in tert-butanol

### Procedure 1.

2.01 g of ezetimibe (hydrated form H) was slurried in tert-butanol (5 ml) at room temperature. While stirred magnetically the mixture thickened considerably within 10 min. The solid was collected by filtration and air-dried for 16 h. Ezetimibe tert-butanol solvate (2.10 g) was obtained in admixture with a trace amount of hydrated form (S>>H) according to XRPD analysis, which melted first at 85.5-90.5° C., resolidified above 106° C. and melted second at 156-160° C. The sample contained 0.51% of water according to KF analysis and showed LOD of −12.8% at 130° C.

### Procedure 2.

2.03 g of ezetimibe (hydrated form H) was slurried in tert-butanol (5 ml) at room temperature. While stirred mechanically the mixture thickened considerably within 45 min. The solid was collected by filtration and air-dried for 20 h. Ezetimibe tert-butanol solvate (2.17 g) was obtained in admixture with a trace amount of hydrated form (S>>H) according to XRPD analysis, which melted first at 87-92° C., resolidified above this temperature and melted second at 160-163° C. The sample contained 0.48% of water according to KF analysis and showed LOD of −12.4% at 130° C.

### Drying of the hydrated form H of ezetimibe

### Example 44

### Water sorption/desorption properties of ezetimibe

Anhydro form of ezetimibe was tested on the automatic water sorption analyzer DVS-1 (Surface Measurement Systems Ltd., London, GB) under the following conditions:
controlled room temperature (25° C.)
nitrogen flow 200 ml/min
two full cycles from 0% RH to 95% RH and back in eleven stages
minimal time per one stage (when dm/dt<0.002%) was 10 min
maximal time per one stage was 360 min.

Results: In the first cycle, no significant absorption of water was observed up to 50% RH. At 60% RH the water sorption affinity became very high and reached equilibrium at 4.2% mass change. At even higher relative humidities the water sorption was only slightly increased (4.4% total at 95% RH). In the desorption cycle the mass was not significantly changed down to 30% RH while it dropped sharply at 20% RH. The second cycle took a similar course except that the water sorption increased sharply already at 50% RH.

### Example 45

Hydrated form H of ezetimibe (from Example 30) was heated in a Mettler HR73 Halogen Moisture Analyzer. Constant value of LOD of −4.5% was achieved after 11.5 min at 50° C., 6.5 min at 60° C., or 4.5 min at 70° C. In all cases anhydro form of ezetimibe resulted according to XRPD analysis.

### Example 46

Hydrated form H of ezetimibe was dried in an air dryer (RH was 35%) at two different temperatures. Drying was very fast at 40° C., when only anhydro form of ezetimibe was detected after 1 h according to XRPD analysis.

### Example 47

Hydrated form H of ezetimibe was dried in a vacuum dryer at an ambient temperature and a pressure of cca. 100 mbar. After 1 h anhydrous form already predominated (A>H), after 2 h it became the sole form according to XRPD analysis.

### Drying of the Ezetimibe Tert-Butanol Solvate

### Example 48

Pure S form of ezetimibe (tert.-butanol solvate; from Example 32) was heated in a Mettler HR73 Halogen Moisture Analyzer at different temperatures for the time indicated and analyzed by XRPD. Results are shown in Table 5.

TABLE 5

| Temperature | | | Resulting |
|---|---|---|---|
| (° C.) | LOD (%) | Time (min) | cryst. form |
| 50 | −0.83 | 14 | S |
| 60 | −1.10 | 10.5 | S |
| 70 | −2.34 | 9 | S |
| 130 | −12.0 | 4.5 | A |

### Preparing Anhydrous Form A of Ezetimibe from Ezetimibe Tert-Butanol Solvate

### Example 49

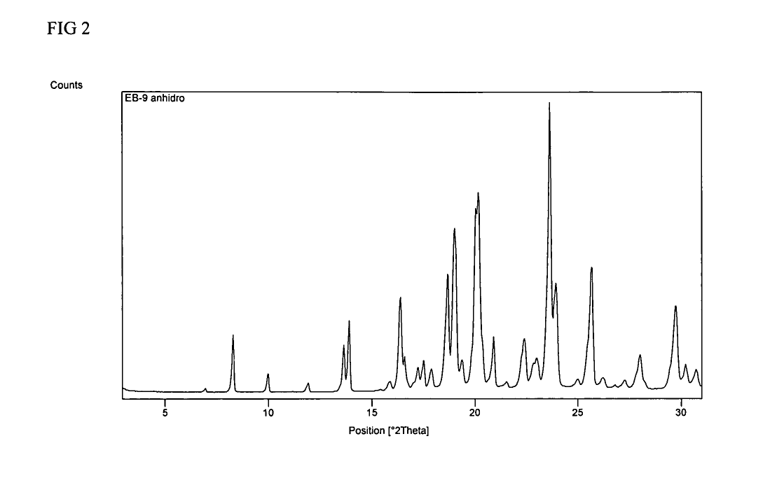

Ezetimibe tert-butanol solvate (5 g) was suspended in mixture of 20 mL water with 5 mL of isopropanol. Suspension was stirred with magnetic stirrer for 45 minutes at 50° C. Product was filtered and dried in vacuum dryer at 50° C. for 10 hours. Anhydrous form of ezetimibe with primary particles of average size 11 μm were obtained. Morphology of particles is shown in FIG. 2.

### Example 50

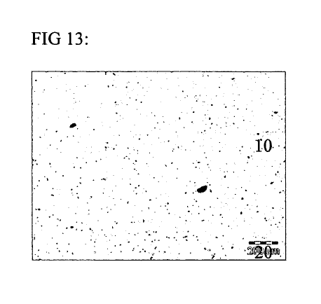

Ezetimibe tert-butanol solvate (1 g) was suspended in 10 mL of water. Suspension was stirred with overhead stirrer for 45 minutes at room temperature. Product was filtered and dried in vacuum dryer at 50° C. for 10 hours. Anhydrous form of ezetimibe with primary particles of average size 4-5 μm were obtained. Anhydrous form of ezetimibe was prepared with primary particles as shown in FIG. 13.

### Example 51

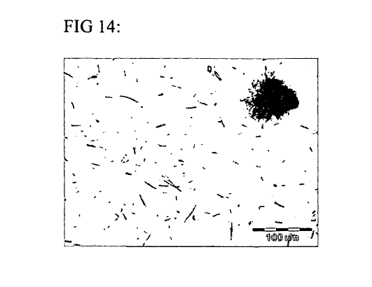

Ezetimibe tert-butanol solvate (1 g) was suspended in mixture of 10 mL of toluene and 0.2 mL acetonitrile. Suspension was stirred with overhead stirrer for 45 minutes at room temperature. Product was filtered and dried in vacuum dryer at 50° C. for 10 hours. Anhydrous form of ezetimibe with morphology of particles as shown in FIG. 14 is obtained.

### Preparing Pharmaceutical Composition Containing Ezetimibe

### Example 52

| | | Comp. | Comp. | Comp. | Comp | Comp |
|---|---|---|---|---|---|---|
| | | A | B | C | D | E |
| 1 | Ezetimibe | 10 | 10 | 10 | 10 | 10 |
| 2 | Lactose | 56.5 | 58.5 | 54.5 | 52.3 | 56.5 |
| | monohydrate | | | | | |
| 3 | Microcrystalline | 20 | 20 | 20 | 20 | |
| | cellulose | | | | | |
| 4 | Povidone K30 | 5 | 3 | 3 | 5 | 5 |
| 5 | Crospovidone | 6 | 6 | 10 | | 6 |
| 6 | Sodium lauryl | 1.5 | 1.5 | 1.5 | 1.5 | 1.5 |
| | sulphate | | | | | |
| 7 | Magnesium stearate | 1 | 1 | 1 | 1 | 1 |
| 8 | Citric acid | | | | 0.25 | |
| 9 | Kollidon CL | | | | 10.0 | |
| 10 | Manitol | | | | | 20 |
| | Total mass (mg) | 100 | 100 | 100 | 100 | 100 |

Mix item 1 in any polymorphic form (corresponding to 10 mg of anhydrous form of) ezetimibe) with water to form a suspension of API and item 8 in water. Add item 4 and optionally item 6 to form granulation solution. Spray the granulation solution and then water over items 2, 3, 6 and portion of 5 in a fluid bed processor to granulate the ingredients. Continue fluidization to dry the damp granules. Screen the dried granules and blend with item 3 and then remainder of item 5. Add item 7 and mix. Compress the mixture to form tablets.

### Examples 53-71

The compositions for examples 53-71 are given in table 7:

| | Example No. | | | | | | | | | |
|---|---|---|---|---|---|---|---|---|---|---|
| | 53 | 54 | 55 | 56 | 57 | 58 | 59 | 60 | 61 | 62 |
| Ingredient | (mg) | (mg) | (mg) | (mg) | (mg) | (mg) | (mg) | (mg) | (mg) | (mg) |
| Ezetimibe | 10 | 10 | 10 | 10 | 10 | 10 | 10 | 10 | 10 | 10 |
| Lactose | 65 | — | — | 53 | 47 | 49 | — | — | — | 57.8 |
| monohydrate | | | | | | | | | | |
| Mannitol | — | — | 52.8 | — | — | — | 54 | — | 50 | — |
| Calcium | — | 55 | — | — | — | — | — | 40 | — | — |
| phosphate | | | | | | | | | | |
| Microcrystalline | 20 | 20 | 25 | 25 | 20 | 30 | 25 | 35 | 30 | 20 |
| cellulose | | | | | | | | | | |
| Povidone | 1 | 4 | 3 | 3 | 3 | — | — | — | — | — |
| K25-K30 | | | | | | | | | | |
| HPC | — | — | — | — | — | 2 | 4 | 4 | 3 | — |
| Klucel EF ® | | | | | | | | | | |
| HPMC | — | — | — | — | — | — | — | — | — | 2 |
| Pharmacoat ®603 | | | | | | | | | | |
| Croscarmellose Na | 1 | 8 | — | — | 6 | 6 | 4 | — | — | 7 |
| (Ac-di-sol) | | | | | | | | | | |
| Sodium starch | — | — | 6 | — | — | — | — | 8 | — | — |
| glycolate | | | | | | | | | | |
| Crospovidone | — | — | — | 5 | — | — | — | — | 4 | — |
| L-HPC | — | — | — | — | 10 | — | — | — | — | — |
| LH-21 | | | | | | | | | | |
| Sodium | 2 | 2 | 2 | 2 | 2 | — | — | 2 | 2 | 2 |
| laurilsulfate | | | | | | | | | | |
| Polysorbate 80 | — | — | — | — | — | 2 | 2 | — | — | — |
| Talc | — | — | — | 1 | 1 | — | — | — | — | — |
| Silica | — | — | 0.2 | — | — | — | — | — | — | 0.2 |
| Magnesium | — | 1 | — | 1 | 1 | — | 1 | 1 | — | 1 |
| stearate | | | | | | | | | | |
| Sodium Stearyl | 1 | — | 1 | — | — | 1 | — | — | 1 | — |
| fumarate | | | | | | | | | | |
| TOTAL | 100 | 100 | 100 | 100 | 100 | 100 | 100 | 100 | 100 | 100 |

| | Example No. | | | | | | | | |
|---|---|---|---|---|---|---|---|---|---|
| | 63 | 64 | 65 | 66 | 67 | 68 | 69 | 70 | 71 |
| Ingredient | (mg) | (mg) | (mg) | (mg) | (mg) | (mg) | (mg) | (mg) | (mg) |
| Ezetimibe | 10 | 10 | 10 | 10 | 10 | 10 | 10 | 10 | 10 |
| Lactose | 53 | — | — | 47.5 | 51 | 49 | — | 55 | 55 |
| monohydrate | | | | | | | | | |
| Mannitol | — | — | 41 | — | — | — | 49 | — | — |
| Calcium | — | 52 | — | — | — | — | — | — | — |
| phosphate | | | | | | | | | |
| Microcrystalline | 20 | 25 | 30 | 30 | 30 | 30 | 29 | 20 | 24 |
| cellulose | | | | | | | | | |
| Povidone | — | — | — | — | 2 | 2 | — | 4 | — |
| K25-K30 | | | | | | | | | |
| HPC | — | — | — | — | — | — | 3 | — | — |
| Klucel EF ® | | | | | | | | | |
| HPMC | 3 | 5 | 4 | 3 | — | — | — | — | 2 |
| Pharmacoat ®603 | | | | | | | | | |
| Croscarmellose Na | — | 5 | — | 6 | 4 | 6 | 8 | 8 | 6 |
| (Ac-di-sol) | | | | | | | | | |
| Sodium starch | 10 | — | — | — | — | — | — | — | — |
| glycolate | | | | | | | | | |
| Crospovidone | — | — | — | — | — | — | — | — | — |
| L-HPC | — | — | 12 | — | — | — | 8 | — | — |
| LH-21 | | | | | | | | | |
| Sodium | 2 | — | 2 | — | — | 2 | 2 | 2 | 2 |
| laurilsulfate | | | | | | | | | |
| Polysorbate 80 | — | 2 | — | 2 | 2 | — | — | — | — |
| Talc | 1 | — | — | — | — | — | — | — | — |
| Silica | — | — | — | 0.2 | — | — | — | — | — |
| Magnesium | — | 1 | 1 | — | — | — | 1 | 1 | — |
| stearate | | | | | | | | | |
| Sodium Stearyl | 1 | — | — | 1.3 | 1 | 1 | — | — | 1 |
| fumarate | | | | | | | | | |
| TOTAL | 100 | 100 | 100 | 100 | 100 | 100 | 100 | 100 | 100 |

For the preparation of pharmaceutical compositions ezetimibe, filler (lactose monohydrate, mannitol or calcium phosphate) and disintegrant (Ac-di-sol, Primojel, L-HPC or crospovidone) are mixed. Binder (povidone, HPC or HPMC) is dissolved in purified water, solubilizing agent (sodium lauril sulfate or polysorbate 80) is added and the obtained granulation mixture is sprayed onto the powder mixture in fluid bed granulator. Alternatively, ezetimibe in any polymorphic form (corresponding to 10 mg of anhydrous form of ezetimibe) is suspended in granulation mixture and sprayed onto powder mixture. Alternatively, ezetimibe in any polymorphic form (corresponding to 10 mg of anhydrous form of ezetimibe) is suspended in water, then solubilizing agent is added to the suspension and finally binder is added. Alternatively, binder is dissolved in water then ezetimibe in any polymorphic form (corresponding to 10 mg of anhydrous form of ezetimibe) is supended in obtained solution and finally solubilizing agent is added to the obtained suspension.

Glidant (talc or silica, colloidal anhydrous) and lubricant are admixed and the obtained mixture is pressed into tablets using appropriate compression tool. Alternatively, only part of disintegrant is added intragranularly and the rest added to granules.

### Examples 72-85

The compositions for the subsequent Ezetimibe 10 mg tablets may be prepared as described previously (cf. example 53-71).

| Example No. | 72 | 73 | 74 | 75 | 76 | 77 | 78 | 79 | 80 | 81 | 82 | 83 | 84 | 85 |
|---|---|---|---|---|---|---|---|---|---|---|---|---|---|---|
| Ezetimibe | 10.0 | 10.0 | 10.0 | 10.0 | 10.0 | 10.0 | 10.0 | 10.0 | 10.0 | 10.0 | 10.0 | 10.0 | 10.0 | 10.0 |
| Sodium lauryl | 1.5 | 2.5 | 1.5 | 1.5 | 1.5 | 1.5 | 1.5 | 2.5 | 1.5 | 1.5 | 1.5 | 1.5 | 1.5 | 1.5 |
| sulphate | | | | | | | | | | | | | | |
| Povidone K30 | 5.0 | 5.0 | 3.0 | 5.0 | 5.0 | 5.0 | 5.0 | 5.0 | 5.0 | 5.0 | 5.0 | 5.0 | 5.0 | 5.0 |
| Lactose | 56.5 | 51.5 | 58.5 | 56.5 | 50.5 | 57.5 | 56.25 | | | | | | | |
| monohydrate | | | | | | | | | | | | | | |
| Crosscarmellose | | 10.0 | | | | 3.0 | | | | | | 4.0 | | |
| sodium | | | | | | | | | | | | | | |
| Crospovidone | 6.0 | | 6.0 | | 10.0 | | | | | | 6.0 | | 6.0 | |
| Microcrystalline | 20.0 | 20.0 | 20 | 20.0 | 20.0 | | 20.0 | | 20.0 | | | | 20.0 | 16.0 |
| cellulose | | | | | | | | | | | | | | |
| Mg-stearate | 1.0 | 1.0 | 1.0 | 1.0 | 1.0 | 1.0 | 1.0 | 1.0 | 1.0 | 1.0 | 1.0 | 1.0 | 1.0 | 1.0 |
| Primojel | | | | 6.0 | | | 6.0 | 10.0 | 6.0 | 10.0 | 4.0 | | | |
| Citric acid | | | | | 2.0 | 2.0 | 0.25 | 0.25 | 0.25 | 0.25 | 0.25 | | 0.25 | 0.25 |
| Mannitol | | | | | | 20.0 | | 51.3 | 56.25 | 72.25 | 72.25 | 66.5 | 50.25 | 50.25 |
| Starch | | | | | | | | 20.0 | | | | | | |
| L-HPC | | | | | | | | | | | | 12.0 | 6.0 | 16.0 |

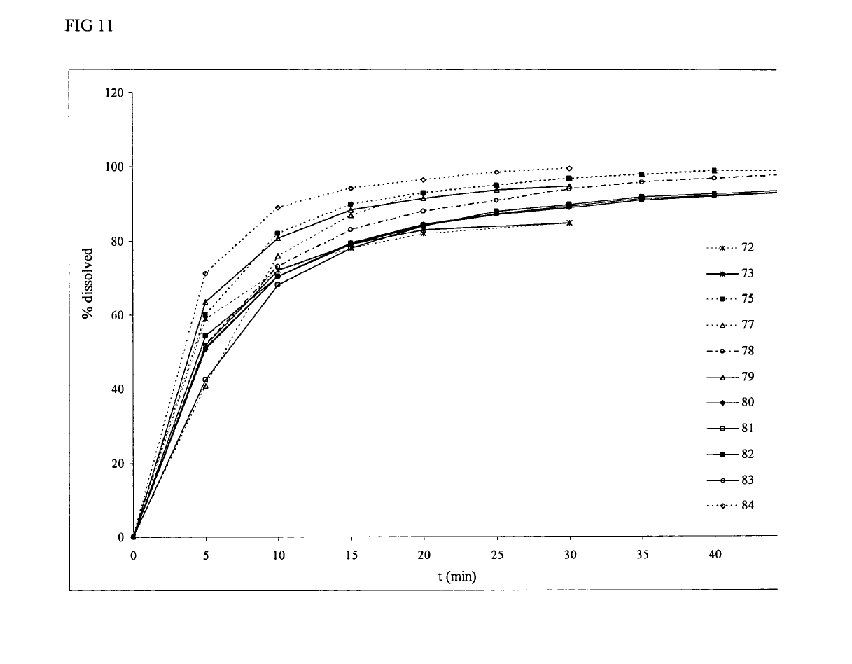

The dissolution profiles (FIG. 11) were prepared by the following way:

1. Dissolution media: 0.1 M HCl with Tween, 900 mL

2. Dissolution Apparatus: Apparatus 2—paddle (Ph.Eur. and USP)

### Example 86

Ezetimibe (4 g) was dissolved in isopropanol (20 mL) at 60° C., then 10 mL of acidified water was added. Further 30 mL of acidified water was dropwise added in 1 hour at 60° C. When addition was completed, the obtained suspension was additionally stirred with overhead stirrer for 1 hour at 50° C., then product was filtered and washed with water. Product was dried in vacuum dryer at 50° C. for 10 hours. Yield: 91%.

1. A process for preparing a compound represented by general formula (I) 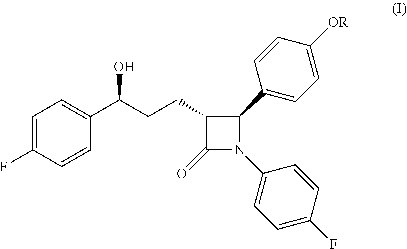
wherein R represents a hydrogen atom, a protective group selected from the group consisting of trisubstituted silyl, arylmethyl, tetrahydro-2H-pyranyl, mono or disubstituted arylmethyl with the substituents, selected from the group consisting of halides, methoxy, nitro, phenyl, naphthyl and any combinations thereof, comprising the steps of
a) metal-catalysed asymmetric transfer hydrogenation of p-fluoroacetophenones of general formula (II) 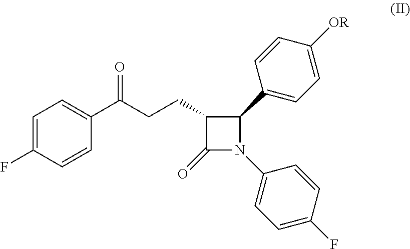
wherein R has the same meaning as above
by using of a hydrogen donor in the presence of a metal catalyst based on Ruthenium complexes
b) obtaining the compound represented by general formula (I). and
c) optionally purifying the compound represented by general formula (I).

2. The process of claim 1, wherein R is selected from the group consisting of t-butyldimethylsilyl, t-butyldiphenylsilyl, triisopropylsilyl, trityl, benzyl, p-bromobenzyl, p-chlorobenzyl, p-nitrobenzyl, o-nitrobenzyl, p-phenylbenzyl, p-methoxybenzyl, tetrahydro-2H-pyranyl characterised in that said process provides the compound represented by general formula (I) with a diastereometric ratio of more than 99:1.

3. The process of claim 1, wherein R is selected from the group consisting of p-bromo-benzyl, p-chlorobenzyl, p-nitrobenzyl, p-methoxy benzyl, trityl, tert-butyldimethylsilyl, tetrahydro-2H-pyranyl and benzyl.

4. The process of claim 1, wherein the metal catalyst is based on Ruthenium complex of optically active N-sulfamoyl-1,2-diamine ligands of the general formula (VI): 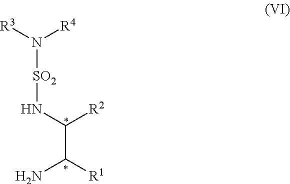
wherein:
C* represents an asymmetric carbon atom;
R¹ and R² independently represent a hydrogen atom, an optionally substituted aryl, or cycloalky; or R¹ and R² may be linked together to form a cyclohexane ring;
R³ and R⁴ independently represent a hydrogen atom, a C_{1-J5} alkyl, linear or branched, optionally substituted with an aryl; or R³ and R⁴ may be linked together to form with the nitrogen atom an optionally substituted C₄₋₆ ring.

5. The process of claim 4 wherein the optically active N-sulfamoy 1-1,2-diamine ligands has enantiomeric excess more than 99%.

6. The process of claim 4 wherein R³ and/or R⁴ can be selected from the group consisting of methyl, iso-propyl and cyclohexyl.

7. The process of claim 4 wherein R³ and R⁴ are linked together to form a ring selected from the group consisting of pyrrolidyl, piperidyl, morpholyl and azepanyl.

8. The process of claim 1 wherein the metal catalyst is prepared from a ruthenium metal precursor and an optically active N-sulfamoy 1-1,2-diamine ligand of the general formula (VI).

9. The process of claim 8 wherein the ruthenium catalyst precursor consists of η⁶-arene-ruthenium(II) halide dimers of the formula [RuX₂(η⁶-arene)]₂, wherein η⁶-arene represents an arene, selected from the group consisting of benzene, p-cymene, mesitylene, 1,3,5-triethylbenzene, hexamethylbenzene and anisole, and X is halide selected from the group consisting of chloride, bromide and iodide.

10. The process according to claim 1 wherein Ruthenium complex is [(S,S)—N-(piperidyl-N-sulfonyl)-1,2-diphenylethylenediamine](η⁶-mesitylene)ruthenium.

11. The process of claim 1 wherein the hydrogen donor is based on HCO₂H.

12. The process of claim 10 wherein the hydrogen donor is selected from the group consisting of HCO₂H-Et₃N, HCO₂H-iso-Pr₂NEt, HCO₂H-metal bicarbonates and HCO₂H-metal carbonates wherein the metal is selected from the group consisting of Na, K, Cs, Mg and Ca.

13. The process of claim 1 wherein the metal-catalysed asymmetric transfer hydrogenation is conducted in solvent selected from the group consisting of dichloroethane, acetonitrile, N,N-dimethyl formamide, N,N-dimethylacetamide, 1-methyl-2-pyrrolidinone (NMP), 1,1,3,3-tetramethylurea, 1,3-dimethyl-2-imidazolidinone, dimethylpropyleneurea and mixtures thereof.

14. The process of claim 1, wherein the compound of formula (I) is ezetimibe.

15.-88. (canceled)
